SEQUENCE LISTING (143 sequences)

SEQ ID NO: 1 - DNA, 21 bp, Artificial Sequence
  Description of Artificial Sequence Synthetic oligonucleotide
gcgcuucaggcacuacaaann

SEQ ID NO: 2 - DNA, 21 bp, Artificial Sequence
  Description of Artificial Sequence Synthetic oligonucleotide
gaagcagacguacuuggcann

SEQ ID NO: 3 - DNA, 21 bp, Artificial Sequence
  Description of Artificial Sequence Synthetic oligonucleotide
cggacuuuagucagaaggann

SEQ ID NO: 4 - DNA, 21 bp, Artificial Sequence
  Description of Artificial Sequence Synthetic oligonucleotide
cccgucaaucaagucuacann

SEQ ID NO: 5 - DNA, 21 bp, Artificial Sequence
  Description of Artificial Sequence Synthetic oligonucleotide
uggccggcgcuucaggcacnn

SEQ ID NO: 6 - DNA, 21 bp, Artificial Sequence
  Description of Artificial Sequence Synthetic oligonucleotide
ccggcgcuucaggcacuacnn

SEQ ID NO: 7 - DNA, 21 bp, Artificial Sequence
  Description of Artificial Sequence Synthetic oligonucleotide
cuucaggcacuacaaauacnn

SEQ ID NO: 8 - DNA, 21 bp, Artificial Sequence
  Description of Artificial Sequence Synthetic oligonucleotide
caggcacuacaaauacugunn

SEQ ID NO: 9 - RNA, 21 nt, Artificial Sequence
  Description of Artificial Sequence Synthetic oligonucleotide
ccgcuucaggcacuacaaaua

SEQ ID NO: 10 - RNA, 21 nt, Artificial Sequence
  Description of Artificial Sequence Synthetic oligonucleotide
gggcuucaggcacuacaaaua

SEQ ID NO: 11 - RNA, 21 nt, Artificial Sequence
  Description of Artificial Sequence Synthetic oligonucleotide
gcccuucaggcacuacaaaua

SEQ ID NO: 12 - RNA, 21 nt, Artificial Sequence
  Description of Artificial Sequence Synthetic oligonucleotide
gcgguucaggcacuacaaaua

SEQ ID NO: 13 - RNA, 21 nt, Artificial Sequence
  Description of Artificial Sequence Synthetic oligonucleotide
gcgcuugaggcacuacaaaua

SEQ ID NO: 14 - RNA, 21 nt, Artificial Sequence
  Description of Artificial Sequence Synthetic oligonucleotide
gcgcuucagccacuacaaaua

SEQ ID NO: 15 - RNA, 21 nt, Artificial Sequence
  Description of Artificial Sequence Synthetic oligonucleotide
gcgcuucagggacuacaaaua

SEQ ID NO: 16 - RNA, 21 nt, Artificial Sequence
  Description of Artificial Sequence Synthetic oligonucleotide
gcgcuucaggcaguacaaaua

SEQ ID NO: 17 - RNA, 21 nt, Artificial Sequence
  Description of Artificial Sequence Synthetic oligonucleotide
gcgcuucaggcacuagaaaua

SEQ ID NO: 18 - RNA, 21 nt, Artificial Sequence
  Description of Artificial Sequence Synthetic oligonucleotide
gcgcuugagccacuacaaaua

SEQ ID NO: 19 - RNA, 21 nt, Artificial Sequence
  Description of Artificial Sequence Synthetic oligonucleotide
gcgcuucagcgacuacaaaua

SEQ ID NO: 20 - RNA, 21 nt, Artificial Sequence
  Description of Artificial Sequence Synthetic oligonucleotide
gcgcuucagccaguacaaaua

SEQ ID NO: 21 - RNA, 21 nt, Artificial Sequence
  Description of Artificial Sequence Synthetic oligonucleotide
gcgcuucagccacuagaaaua

SEQ ID NO: 22 - RNA, 21 nt, Artificial Sequence
  Description of Artificial Sequence Synthetic oligonucleotide
gcgcuucaggcacuacaaaua

SEQ ID NO: 23 - RNA, 21 nt, Artificial Sequence
  Description of Artificial Sequence Synthetic oligonucleotide
gcgcuucaggcacuacaaaua

SEQ ID NO: 24 - RNA, 21 nt, Artificial Sequence
  Description of Artificial Sequence Synthetic oligonucleotide
gcgcuucaggcacuacaaaua

SEQ ID NO: 25 - RNA, 21 nt, Artificial Sequence
  Description of Artificial Sequence Synthetic oligonucleotide
gcgcuucaggcacuacaaaua

SEQ ID NO: 26 - RNA, 21 nt, Artificial Sequence
  Description of Artificial Sequence Synthetic oligonucleotide
gcgcuucaggcacuacaaaua

SEQ ID NO: 27 - RNA, 21 nt, Artificial Sequence
  Description of Artificial Sequence Synthetic oligonucleotide
gcgcuucaggcacuacaaaua

SEQ ID NO: 28 - RNA, 21 nt, Artificial Sequence
  Description of Artificial Sequence Synthetic oligonucleotide
gcgcuucaggcacuacaaaua

SEQ ID NO: 29 - RNA, 21 nt, Artificial Sequence
  Description of Artificial Sequence Synthetic oligonucleotide
gcgcuucaggcacuacaaaua

SEQ ID NO: 30 - RNA, 21 nt, Artificial Sequence
  Description of Artificial Sequence Synthetic oligonucleotide
gcgcuucaggcacuacaaaua

SEQ ID NO: 31 - RNA, 21 nt, Artificial Sequence
  Description of Artificial Sequence Synthetic oligonucleotide
gcgcuucaggcacuacaaaua

SEQ ID NO: 32 - RNA, 21 nt, Mus musculus
caggcauuccagagaaagcgu

SEQ ID NO: 33 - RNA, 21 nt, Mus musculus
gcauuccagagaaagcguuua

SEQ ID NO: 34 - RNA, 21 nt, Mus musculus
cagauaagugaucgaucuaga

SEQ ID NO: 35 - RNA, 21 nt, Mus musculus
agugcuucucgaccacagaca

SEQ ID NO: 36 - RNA, 21 nt, Mus musculus
uuccagagaaagcguuuaauu

SEQ ID NO: 37 - RNA, 21 nt, Mus musculus
cagagaaagcguuuaauuuaa

SEQ ID NO: 38 - RNA, 21 nt, Artificial Sequence
  Description of Artificial Sequence Synthetic oligonucleotide
uuuguagugccugaagcgccg

SEQ ID NO: 39 - RNA, 21 nt, Artificial Sequence
  Description of Artificial Sequence Synthetic oligonucleotide
uuuguagugccugaagcgccg

SEQ ID NO: 40 - RNA, 21 nt, Artificial Sequence
  Description of Artificial Sequence Synthetic oligonucleotide
uuuguagugccugaagcgccg

SEQ ID NO: 41 - RNA, 21 nt, Artificial Sequence
  Description of Artificial Sequence Synthetic oligonucleotide
uuuguagugccugaagcgccg

SEQ ID NO: 42 - RNA, 21 nt, Artificial Sequence
  Description of Artificial Sequence Synthetic oligonucleotide
uuuguagugccugaagcgccg

SEQ ID NO: 43 - RNA, 21 nt, Artificial Sequence
  Description of Artificial Sequence Synthetic oligonucleotide
uuuguagugccugaagcgccg

SEQ ID NO: 44 - RNA, 21 nt, Artificial Sequence
  Description of Artificial Sequence Synthetic oligonucleotide
uuuguagugccugaagcgccg

SEQ ID NO: 45 - RNA, 21 nt, Artificial Sequence
  Description of Artificial Sequence Synthetic oligonucleotide
uuuguagugccugaagcgccg

SEQ ID NO: 46 - RNA, 21 nt, Artificial Sequence
  Description of Artificial Sequence Synthetic oligonucleotide
uuuguagugccugaagcgccg

SEQ ID NO: 47 - RNA, 21 nt, Artificial Sequence
  Description of Artificial Sequence Synthetic oligonucleotide
uuuguagugccugaagcgccg

SEQ ID NO: 48 - RNA, 21 nt, Mus musculus
gcuuucucuggaaugccugca

SEQ ID NO: 49 - RNA, 21 nt, Mus musculus
aacgcuuucucuggaaugccu

SEQ ID NO: 50 - RNA, 21 nt, Mus musculus
uagaucguacacuuaucugua

SEQ ID NO: 51 - RNA, 21 nt, Mus musculus
ucuguggucgagaagcacuug

SEQ ID NO: 52 - RNA, 21 nt, Mus musculus
uuaaacgcuuucucuggaaug

SEQ ID NO: 53 - RNA, 21 nt, Mus musculus
aaauuaaacgcuuucucugga

SEQ ID NO: 54 - DNA, 21 bp, Artificial Sequence
  Description of Artificial Sequence Synthetic oligonucleotide
uuuguagugccugaagcgcnn

SEQ ID NO: 55 - DNA, 21 bp, Artificial Sequence
  Description of Artificial Sequence Synthetic oligonucleotide
ugccaaguacgucugcuucnn

SEQ ID NO: 56 - DNA, 21 bp, Artificial Sequence
  Description of Artificial Sequence Synthetic oligonucleotide
uccuucugacuaaaguccgnn

SEQ ID NO: 57 - DNA, 21 bp, Artificial Sequence
  Description of Artificial Sequence Synthetic oligonucleotide
uguagacuugauugacgggnn

SEQ ID NO: 58 - DNA, 21 bp, Artificial Sequence
  Description of Artificial Sequence Synthetic oligonucleotide
gugccugaagcgccggccann

SEQ ID NO: 59 - DNA, 21 bp, Artificial Sequence
  Description of Artificial Sequence Synthetic oligonucleotide
guagugccugaagcgccggnn

SEQ ID NO: 60 - DNA, 21 bp, Artificial Sequence
  Description of Artificial Sequence Synthetic oligonucleotide
guauuuguagugccugaagnn

SEQ ID NO: 61 - DNA, 21 bp, Artificial Sequence
  Description of Artificial Sequence Synthetic oligonucleotide
acauuuguagugccugaagnn

SEQ ID NO: 62 - RNA, 21 nt, Artificial Sequence
  Description of Artificial Sequence Synthetic oligonucleotide
uuuguagugccugaagcggcg

SEQ ID NO: 63 - RNA, 21 nt, Artificial Sequence
  Description of Artificial Sequence Synthetic oligonucleotide
uuuguagugccugaagccccg

SEQ ID NO: 64 - RNA, 21 nt, Artificial Sequence
  Description of Artificial Sequence Synthetic oligonucleotide
uuuguagugccugaagggccg

SEQ ID NO: 65 - RNA, 21 nt, Artificial Sequence
  Description of Artificial Sequence Synthetic oligonucleotide
uuuguagugccugaaccgccg

SEQ ID NO: 66 - RNA, 21 nt, Artificial Sequence
  Description of Artificial Sequence Synthetic oligonucleotide
uuuguagugccucaagcgccg

SEQ ID NO: 67 - RNA, 21 nt, Artificial Sequence
  Description of Artificial Sequence Synthetic oligonucleotide
uuuguaguggcugaagcgccg

SEQ ID NO: 68 - RNA, 21 nt, Artificial Sequence
  Description of Artificial Sequence Synthetic oligonucleotide
uuuguagucccugaagcgccg

SEQ ID NO: 69 - RNA, 21 nt, Artificial Sequence
  Description of Artificial Sequence Synthetic oligonucleotide
uuuguacugccugaagcgccg

SEQ ID NO: 70 - RNA, 21 nt, Artificial Sequence
  Description of Artificial Sequence Synthetic oligonucleotide
uuucuagugccugaagcgccg

SEQ ID NO: 71 - RNA, 21 nt, Artificial Sequence
  Description of Artificial Sequence Synthetic oligonucleotide
uuuguaguggcucaagcgccg

SEQ ID NO: 72 - RNA, 21 nt, Artificial Sequence
  Description of Artificial Sequence Synthetic oligonucleotide
uuuguagucgcugaagcgccg

SEQ ID NO: 73 - RNA, 21 nt, Artificial Sequence
  Description of Artificial Sequence Synthetic oligonucleotide
uuuguacuggcugaagcgccg

SEQ ID NO: 74 - RNA, 21 nt, Artificial Sequence
  Description of Artificial Sequence Synthetic oligonucleotide
uuucuaguggcugaagcgccg

SEQ ID NO: 75 - DNA, 21 bp, Artificial Sequence
  Description of Artificial Sequence Synthetic oligonucleotide
gcgcuucaggcacuacaaatt

SEQ ID NO: 76 - DNA, 21 bp, Artificial Sequence
  Description of Artificial Sequence Synthetic oligonucleotide
uuuguagugccugaagcgctt

SEQ ID NO: 77 - DNA, 21 bp, Artificial Sequence
  Description of Artificial Sequence Synthetic oligonucleotide
gaagcagacguacuuggcatt

SEQ ID NO: 78 - DNA, 21 bp, Artificial Sequence
  Description of Artificial Sequence Synthetic oligonucleotide
ugccaaguacgucugcuuctt

SEQ ID NO: 79 - DNA, 21 bp, Artificial Sequence
  Description of Artificial Sequence Synthetic oligonucleotide
cggacuuuagucagaaggatt

SEQ ID NO: 80 - DNA, 21 bp, Artificial Sequence
  Description of Artificial Sequence Synthetic oligonucleotide
uccuucugacuaaaguccgtt

SEQ ID NO: 81 - DNA, 21 bp, Artificial Sequence
  Description of Artificial Sequence Synthetic oligonucleotide
cccgucaaucaagucuacatt

SEQ ID NO: 82 - DNA, 21 bp, Artificial Sequence
  Description of Artificial Sequence Synthetic oligonucleotide
uguagacuugauugacgggtt

SEQ ID NO: 83 - DNA, 21 bp, Artificial Sequence
  Description of Artificial Sequence Synthetic oligonucleotide
uggccggcgcuucaggcactt

SEQ ID NO: 84 - DNA, 21 bp, Artificial Sequence
  Description of Artificial Sequence Synthetic oligonucleotide
gugccugaagcgccggccatt

SEQ ID NO: 85 - DNA, 21 bp, Artificial Sequence
  Description of Artificial Sequence Synthetic oligonucleotide
ccggcgcuucaggcacuactt

SEQ ID NO: 86 - DNA, 21 bp, Artificial Sequence
  Description of Artificial Sequence Synthetic oligonucleotide
guagugccugaagcgccggtt

SEQ ID NO: 87 - DNA, 21 bp, Artificial Sequence
  Description of Artificial Sequence Synthetic oligonucleotide
cuucaggcacuacaaauactt

SEQ ID NO: 88 - DNA, 21 bp, Artificial Sequence
  Description of Artificial Sequence Synthetic oligonucleotide
guauuuguagugccugaagtt

SEQ ID NO: 89 - DNA, 21 bp, Artificial Sequence
  Description of Artificial Sequence Synthetic oligonucleotide
caggcacuacaaauacugutt

SEQ ID NO: 90 - DNA, 21 bp, Artificial Sequence
  Description of Artificial Sequence Synthetic oligonucleotide
acaguauuuguagugccugtt

SEQ ID NO: 91 - DNA, 21 bp, Artificial Sequence
  Description of Artificial Sequence Synthetic oligonucleotide
uaccuggagacaaaccucgtt

SEQ ID NO: 92 - DNA, 21 bp, Artificial Sequence
  Description of Artificial Sequence Synthetic oligonucleotide
cgagguuugucuccagguatt

SEQ ID NO: 93 - DNA, 21 bp, Artificial Sequence
  Description of Artificial Sequence Synthetic oligonucleotide
gcuggaaggagaacucccctt

SEQ ID NO: 94 - DNA, 21 bp, Artificial Sequence
  Description of Artificial Sequence Synthetic oligonucleotide
ggggaguucuccuuccagctt

SEQ ID NO: 95 - DNA, 21 bp, Artificial Sequence
  Description of Artificial Sequence Synthetic oligonucleotide
cuccccagaguucacaccutt

SEQ ID NO: 96 - DNA, 21 bp, Artificial Sequence
  Description of Artificial Sequence Synthetic oligonucleotide
aggugugaacucuggggagtt

SEQ ID NO: 97 - DNA, 21 bp, Artificial Sequence
  Description of Artificial Sequence Synthetic oligonucleotide
gcacuacaaauacuguggctt

SEQ ID NO: 98 - DNA, 21 bp, Artificial Sequence
  Description of Artificial Sequence Synthetic oligonucleotide
gccacaguauuuguagugctt

SEQ ID NO: 99 - DNA, 21 bp, Artificial Sequence
  Description of Artificial Sequence Synthetic oligonucleotide
agguggccggcgcuucaggtt

SEQ ID NO: 100 - DNA, 21 bp, Artificial Sequence
  Description of Artificial Sequence Synthetic oligonucleotide
ccugaagcgccggccaccutt

SEQ ID NO: 101 - DNA, 46 bp, Artificial Sequence
  Description of Artificial Sequence Synthetic oligonucleotide
tggccggcgcttcaggcactacaaatactgtggcagcatataattt

SEQ ID NO: 102 - DNA, 40 bp, Artificial Sequence
  Description of Artificial Sequence Synthetic oligonucleotide
tgaccgcgggtgcaggcattccagagaaagcgtttaattt

SEQ ID NO: 103 - DNA, 21 bp, Artificial Sequence
  Description of Artificial Sequence Synthetic oligonucleotide
gcgcuucaggcacuacaaatt

SEQ ID NO: 104 - DNA, 21 bp, Artificial Sequence
  Description of Artificial Sequence Synthetic oligonucleotide
gaagcagacguacuuggcatt

SEQ ID NO: 105 - DNA, 21 bp, Artificial Sequence
  Description of Artificial Sequence Synthetic oligonucleotide
cggacuuuagucagaaggatt

SEQ ID NO: 106 - DNA, 21 bp, Artificial Sequence
  Description of Artificial Sequence Synthetic oligonucleotide
cccgucaaucaagucuacatt

SEQ ID NO: 107 - DNA, 21 bp, Artificial Sequence
  Description of Artificial Sequence Synthetic oligonucleotide
uggccggcgcuucaggcactt

SEQ ID NO: 108 - DNA, 21 bp, Artificial Sequence
  Description of Artificial Sequence Synthetic oligonucleotide
ccggcgcuucaggcacuactt

SEQ ID NO: 109 - DNA, 21 bp, Artificial Sequence
  Description of Artificial Sequence Synthetic oligonucleotide
cuucaggcacuacaaauactt

SEQ ID NO: 110 - DNA, 21 bp, Artificial Sequence
  Description of Artificial Sequence Synthetic oligonucleotide
caggcacuacaaauacugutt

SEQ ID NO: 111 - DNA, 21 bp, Artificial Sequence
  Description of Artificial Sequence Synthetic oligonucleotide
ccgcuucaggcacuacaaaua

SEQ ID NO: 112 - DNA, 21 bp, Artificial Sequence
  Description of Artificial Sequence Synthetic oligonucleotide
gggcuucaggcacuacaaaua

SEQ ID NO: 113 - DNA, 21 bp, Artificial Sequence
  Description of Artificial Sequence Synthetic oligonucleotide
gcccuucaggcacuacaaaua

SEQ ID NO: 114 - DNA, 21 bp, Artificial Sequence
  Description of Artificial Sequence Synthetic oligonucleotide
gcgguucaggcacuacaaaua

SEQ ID NO: 115 - DNA, 21 bp, Artificial Sequence
  Description of Artificial Sequence Synthetic oligonucleotide
gcgcuugaggcacuacaaaua

SEQ ID NO: 116 - DNA, 21 bp, Artificial Sequence
  Description of Artificial Sequence Synthetic oligonucleotide
gcgcuucagccacuacaaaua

SEQ ID NO: 117 - DNA, 21 bp, Artificial Sequence
  Description of Artificial Sequence Synthetic oligonucleotide
gcgcuucagggacuacaaaua

SEQ ID NO: 118 - DNA, 21 bp, Artificial Sequence
  Description of Artificial Sequence Synthetic oligonucleotide
gcgcuucaggcaguacaaaua

SEQ ID NO: 119 - DNA, 21 bp, Artificial Sequence
  Description of Artificial Sequence Synthetic oligonucleotide
gcgcuucaggcacuagaaaua

SEQ ID NO: 120 - DNA, 21 bp, Artificial Sequence
  Description of Artificial Sequence Synthetic oligonucleotide
gcgcuugagccacuacaaaua

SEQ ID NO: 121 - DNA, 21 bp, Artificial Sequence
  Description of Artificial Sequence Synthetic oligonucleotide
gcgcuucagcgacuacaaaua

SEQ ID NO: 122 - DNA, 21 bp, Artificial Sequence
  Description of Artificial Sequence Synthetic oligonucleotide
gcgcuucagccaguacaaaua

SEQ ID NO: 123 - DNA, 21 bp, Artificial Sequence
  Description of Artificial Sequence Synthetic oligonucleotide
gcgcuucagccacuagaaaua

SEQ ID NO: 124 - DNA, 21 bp, Artificial Sequence
  Description of Artificial Sequence Synthetic oligonucleotide
gcgcuucaggcacuacaaaua

SEQ ID NO: 125 - DNA, 21 bp, Artificial Sequence
  Description of Artificial Sequence Synthetic oligonucleotide
gcgcuucaggcacuacaaaua

SEQ ID NO: 126 - DNA, 21 bp, Artificial Sequence
  Description of Artificial Sequence Synthetic oligonucleotide
gcgcuucaggcacuacaaaua

SEQ ID NO: 127 - DNA, 21 bp, Artificial Sequence
  Description of Artificial Sequence Synthetic oligonucleotide
gcgcuucaggcacuacaaaua

SEQ ID NO: 128 - DNA, 21 bp, Artificial Sequence
  Description of Artificial Sequence Synthetic oligonucleotide
gcgcuucaggcacuacaaaua

SEQ ID NO: 129 - DNA, 21 bp, Artificial Sequence
  Description of Artificial Sequence Synthetic oligonucleotide
gcgcuucaggcacuacaaaua

SEQ ID NO: 130 - DNA, 21 bp, Artificial Sequence
  Description of Artificial Sequence Synthetic oligonucleotide
gcgcuucaggcacuacaaaua

SEQ ID NO: 131 - DNA, 21 bp, Artificial Sequence
  Description of Artificial Sequence Synthetic oligonucleotide
gcgcuucaggcacuacaaaua

SEQ ID NO: 132 - DNA, 21 bp, Artificial Sequence
  Description of Artificial Sequence Synthetic oligonucleotide
gcgcuucaggcacuacaaaua

SEQ ID NO: 133 - DNA, 21 bp, Artificial Sequence
  Description of Artificial Sequence Synthetic oligonucleotide
gcgcuucaggcacuacaaaua

SEQ ID NO: 134 - DNA, 21 bp, Artificial Sequence
  Description of Artificial Sequence Synthetic oligonucleotide
uuuguagugccugaagcgccg

SEQ ID NO: 135 - DNA, 21 bp, Artificial Sequence
  Description of Artificial Sequence Synthetic oligonucleotide
uuuguagugccugaagcgccg

SEQ ID NO: 136 - DNA, 21 bp, Artificial Sequence
  Description of Artificial Sequence Synthetic oligonucleotide
uuuguagugccugaagcgccg

SEQ ID NO: 137 - DNA, 21 bp, Artificial Sequence
  Description of Artificial Sequence Synthetic oligonucleotide
uuuguagugccugaagcgccg

SEQ ID NO: 138 - DNA, 21 bp, Artificial Sequence
  Description of Artificial Sequence Synthetic oligonucleotide
uuuguagugccugaagcgccg

SEQ ID NO: 139 - DNA, 21 bp, Artificial Sequence
  Description of Artificial Sequence Synthetic oligonucleotide
uuuguagugccugaagcgccg

SEQ ID NO: 140 - DNA, 21 bp, Artificial Sequence
  Description of Artificial Sequence Synthetic oligonucleotide
uuuguagugccugaagcgccg

SEQ ID NO: 141 - DNA, 21 bp, Artificial Sequence
  Description of Artificial Sequence Synthetic oligonucleotide
uuuguagugccugaagcgccg

SEQ ID NO: 142 - DNA, 21 bp, Artificial Sequence
  Description of Artificial Sequence Synthetic oligonucleotide
uuuguagugccugaagcgccg

SEQ ID NO: 143 - DNA, 21 bp, Artificial Sequence
  Description of Artificial Sequence Synthetic oligonucleotide
uuuguagugccugaagcgccg

(19) United States
(12) Patent Application Publication
(10) Pub. No.: US 2010/0273998 A1
(43) Pub. Date: Oct. 28, 2010

(54) METHODS AND COMPOSITIONS FOR MODULATING TISSUE FACTOR

(76) Inventors: HANS PRYDZ, OSLO (NO); MOHAMMED AMARZGUIOUI, OLSO (NO); TORGEIR HOLEN, OSLO (NO)

Correspondence Address: CLARK & ELBING LLP, 101 FEDERAL STREET, BOSTON, MA, 02110, US

(21) Appl. No.: 12/710,192

(22) Filed: Feb. 22, 2010

Related U.S. Application Data
(63) Continuation of application No. 10/911,947, filed on Aug. 5, 2004, now abandoned.
Application No. 10/911,947 is a continuation-in-part of application No. PCT/NO03/00045, filed on Feb. 6, 2003.
(60) Provisional application No. 60/354,515, filed on Feb. 8, 2002.
(60) Provisional application No. 60/497,314, filed on Aug. 25, 2003.

(30) Foreign Application Priority Data
Feb. 7, 2002 (NO) 20020612
Oct. 16, 2002 (NO) 20024987
Aug. 6, 2003 (NO) 20033492

(51) Int. Cl. C07H 21/02

(52) U.S. Cl. 536/24.5

(57) ABSTRACT

The present invention features nucleic acid molecules, specifically short interfering RNA molecules, that are able to modulate the expression of TF and methods for using such molecules for the treatment of coagulative disorders and for the prevention and treatment of cancer.

### CROSS-REFERENCE TO RELATED APPLICATIONS

This application is a continuation of U.S. patent application Ser. No. 10/911,947, filed Aug. 5, 2004, which is a continuation-in-part application of International Application No. PCT/NO03/00045, filed Feb. 6, 2003, and published in English, which claims the benefit of Norwegian application 20024987, filed on Oct. 16, 2002; U.S. Provisional Application No. 60/354,515, filed on Feb. 8, 2002; and Norwegian Application No. 20020612, filed on Feb. 7, 2002. This application also claims the benefit of U.S. Provisional Application No. 60/497,314, filed on Aug. 25, 2003, and Norwegian Application No. 20033492, filed on Aug. 6, 2003. Each of these applications are incorporated herein by reference.

### SEQUENCE LISTING

The instant application contains a Sequence Listing which has been submitted via EFS-Web and is hereby incorporated by reference in its entirety. Said ASCII copy, created on Feb. 22, 2010, is named 50340_{—}002002.txt, and is 51,215 bytes in size.

### BACKGROUND OF THE INVENTION

The present invention features methods and composition for modulating the expression of Tissue Factor (TF).

TF is a potent trigger of blood coagulation. TF is a membrane-bound glycoprotein that is not normally found soluble in the circulation or accessible to plasma proteins, including factor VII/VIIa, and the other coagulation factors. Expression of TF in the vascular compartment typically results in disseminated intravascular coagulation or localized initiation of clotting.

TF is constitutively expressed on the surface of some extravascular cells in culture including fibroblasts, some as yet unidentified types of brain cells, and certain epithelial cells that are separated from the circulating plasma proteins by basement membrane barriers. The presence of TF on these cells results in clot formation upon contact with blood as a result of tissue damage. Thus, TF is the foundation upon which the hemostatic system is initiated. While blood clotting is a normal physiological process that prevents unwanted blood loss, excessive blood clotting can contribute to various pathological processes including thrombosis following cardiac surgery, thrombolytic disorders, thromboembolic disorders, coagulopathies, and restenosis. TF is instrumental in causing arterial thrombosis upon rupture of atherosclerotic plaques. Furthermore, TF is of central pathogenic importance in cases of septic disseminated intravascular coagulation (e.g., meningococcal sepsis). Given the central role of TF in thrombosis, there is a need for methods to modulate or silence TF activity, allowing for regulation of blood clotting in pathological processes.

In addition to the role of TF in the initiation of normal or pathologic coagulation cascades, there is also some evidence that a high content of TF in cancer cells correlates with cancer-driven angiogenesis and metastasis of the primary tumor. At present, cancer remains a major cause of death and this is often a consequence of metastasis. In the process of metastasis, tumour colonies are established by malignant cells which have detached from the original tumour (primary tumour) and spread throughout the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. Although the primary tumour can often be eliminated by surgery, there is always a risk that metastatic deposits already may exist or may develop due to remnants of the primary tumour after the surgical intervention.

The evidence for the role of TF in tumor metastasis is controversial with some studies suggesting a strong role and other studies suggesting that there is no relationship between TF levels and tumor metastasis. There is, therefore, a need for methods to modulate or silence TF expression, which can be used to investigate the role of TF in metastasis, and to prevent metastasis and provide an effective treatment of cancer patients.

Mechanisms that silence unwanted gene expression are critical for normal cellular function. It has been known for a long time that interactions between homologous DNA and/or RNA sequences can silence genes and induce DNA methylation. The discovery of RNA interference (RNAi) in C. elegans in 1998 focused attention on double-stranded RNA (dsRNA) as an elicitor of gene silencing, and many gene-silencing effects in plants are now known to be mediated by dsRNA.

RNAi is usually described as a post-transcriptional gene-silencing (PTGS) phenomenon in which dsRNAs trigger degradation of homologous mRNA in the cytoplasm. However, the potential of nuclear dsRNA to enter a pathway leading to epigenetic modifications of homologous DNA sequences and silencing at the transcriptional level should not be discounted. Also, even though the nuclear aspects of RNA silencing have been studied primarily in plants, there are indications that similar RNA-directed DNA or chromatin modifications might occur in other organisms as well.

RNAi in animals and the related phenomenon of PTGS in plants are derived from the same highly conserved mechanism, indicating an ancient origin. The basic process involves a dsRNA that is processed into shorter units (called short interfering RNAs (siRNAs)) that guide recognition and targeted cleavage of homologous messenger RNA (mRNA). The dsRNAs that (after processing) trigger RNAi/PTGS can be made in the nucleus or cytoplasm in a number of ways.

The processing of dsRNA into siRNAs, which in turn degrade mRNA, is a two-step RNA degradation process. The first step involves a dsRNA endonuclease (ribonuclease III-like; RNase III-like) activity that processes dsRNA into sense and antisense RNAs which are 21 to 25 nucleotides (nt) long (i.e., siRNA). In Drosophila, this RNase III-type protein is termed Dicer. In the second step, the antisense siRNAs produced combine with, and serve as guides for, a different ribonuclease complex called RNA-induced silencing complex (RISC), which cleaves the homologous single-stranded mRNAs. RISC cuts the mRNA approximately in the middle of the region paired with the antisense siRNA, after which the mRNA is further degraded.

dsRNAs from different sources can enter the processing pathway leading to RNAi/PTGS. Furthermore, recent work also suggests that there may be more than one pathway for dsRNA cleavage, producing distinct classes of siRNAs that may not be functionally equivalent.

RNA silencing (which is active at different levels of gene expression in the cytoplasm and the nucleus) appears to have evolved to counter the proliferation of foreign sequences such as transposable elements and viruses (many of which produce dsRNA during replication). However, as RNAi/PTGS produce a mobile signal that induces silencing at distant sites in lower organisms, the possibility of injecting directly siRNAs to shut down protein synthesis and/or function as a therapeutic tool in mammalian cells should be considered. In general, RNAi holds great potential for applications requiring gene silencing.

### SUMMARY OF THE INVENTION

The present invention relates to compositions and methods using RNA interference to modulate TF expression. These methods and compositions are useful for the treatment and prevention of coagulation disorders and tumor metastasis. Apart from preliminary studies on antibodies, no effective inhibitors that regulate TF gene expression are available. It is therefore an object of the present invention to provide siRNA molecules that, together with RISC, are able to directly modulate the expression of TF in mammals. Generally, the present invention relates to siRNA molecules, which are double or single stranded and comprise at least 21-25 nucleotides, and are able to modulate the gene expression of TF.

The present invention features siRNAs that can bypass the RNAse III-like RNAi initiator Dicer and directly charge the effector nuclease RISC so that TF mRNA is degraded. In addition, given our finding that different siRNAs against the same target vary in efficiency, we have synthesized siRNAs against different parts of TF mRNA, which can then combine with RISC to direct the specific degradation or silencing of TF mRNA.

Also, the siRNA of the present invention may comprise one or two base-pairing mutations compared to the wild type sequence of the present siRNA molecules. For example the modified siRNA molecules are about 90% homologous to the wild type siRNA molecules of the present invention.

The present invention also features siRNA molecules which are chemically modified compared to the wild type siRNA of the present invention.

The present invention further discloses that siRNA silencing is relatively stable but declines over time and that TF coagulation activity can be reduced five-to-ten-fold and remain so over a period of 5 days (120 hours) after a single siRNA transfection. Thus, the present invention also relates to a pharmaceutical preparation comprising the siRNAs of the present invention, as well as use of the pharmaceutical preparation. The present inventors have also found that siRNA molecules directed towards TF can be used to treat, prevent, or inhibit unwanted blood coagulation or a coagulation disorder or to reduce the abilities of the malignant cells to settle and form new tumours in vivo in a mouse model. Thus, the siRNA molecules directed towards TF may be useful in the prevention of coagulation disorders and of metastasis and treatment of cancer in vertebrates, preferably mammals, more preferably humans.

In one aspect, the invention generally features an isolated siRNA molecule, of at least 19 nucleotides, having at least one strand that is substantially complementary to at least ten but no more than thirty consecutive nucleotides of TF, and that reduces the expression of TF gene or protein.

In preferred embodiments, the siRNA molecule has at least one strand that is substantially complementary to at least ten but no more than thirty consecutive nucleotides of nucleotides 160 to 194 of human TF (GenBank No. M16553). More desirable, the isolated siRNA moelcuel has at least one strand that is substantially complementary to 19 to 25 nucleotides of nucleotides 160 to 194 of human TF.

The siRNA molecule can be double-stranded or single-stranded. In another embodiment, the siRNA molecule is at least 19 nucleotides in length, preferably 21 to 25 nucleotides in length, most preferably 21 nucleotides in length. In preferred embodiments, the siRNA molecules can induce cleavage of mRNA.

In preferred embodiments, the tissue factor is a vertebrate or mammalian tissue factor (e.g., human or mouse). In preferred embodiments, the siRNA molecule includes a sequence that is at least 90% homologous, preferably 95%, 99%, or 100% homologous, to a sequence selected from SEQ ID NOs: 1 to 8 (54 to 61) and 32 to 37 (48 to 53), wherein the numbers in parenthesis are used throughout the specification to represent the SEQ ID NOs of the complementary (or antisense) strands. In one preferred embodiment the siRNA molecule includes the sequence set forth in SEQ ID NO: 1 (54) or SEQ ID NO: 2 (55). In additional preferred embodiments, the siRNA molecule includes at least 14 consecutive nucleotides that are at least 90% homologous, preferably 95%, 99%, or 100% homologous to any of SEQ ID NOs: 1 to 8 (54 to 61) or 32 to 37 (48 to 53), and can further include up to 7 nucleotides complementary to nucleotides in human TF that are adjacent to nucleotides in human TF that are complementary to the sequence of any of SEQ ID NOs: 1 to 8 (54 to 61) or 32 to 37 (48 to 53).

The present invention also features siRNA molecules that are modified compared to the siRNA molecule that is complementary to a portion of tissue factor. Modifications can include mutations or substitutions as compared to the siRNA molecule that is perfectly complementary to a portion of tissue factor. Modifications can also include chemical modifications such as the addition of a C1-C3 alkyl group, a C1-C3 alkenyl group, or a C1-C3 alkylyl group in one or more of the 2′OH groups of the siRNA molecule or the introduction of a lower alkyl, such as methyl in the 2′OH-position of the siRNA molecule. Another preferred modification is the replacement of at least one phosphodiester linkage with a phosphorothioate linkage in the sequence. Another preferred modification is allylation. Yet another preferred modification is the inclusion of a 3′ overhang. Overhangs can include any nucleobase (DNA or RNA), or any modified nucleobase, or any combination thereof. Overhangs can include nucleobases that basepair with nucleotides of tissue factor adjacent to the target sequence or that do not basepair with nucleotides of tissue factor adjacent to the target sequence. Non-limiting examples of overhangs include: TT, TA, AT, GC, CG, CC, GG, AA, AC, CA, AG, GA, TC, TG, CT, GT, UU, UA, AU, UC, UG, CU, and GU. Preferred modified siRNA molecules include sequences that are at least 90% homologous, preferably 95%, 99%, or 100% homologous to the any of the sequences set forth in SEQ ID NOs: 9 to 21 (62 to 74) and 22 to 31 (38 to 47). In one embodiment, the siRNA molecules of the present invention comprise a sequence as depicted in SEQ ID NOs: 1 to 8, wherein the phosphodiester bond has been replaced by a thiophosphodiester bond. In preferred embodiments, the modified siRNA sequences include a sequence set forth in SEQ ID NOs: 9 to 11 (62-64), 24 (40), 28 (44), 29 (45).

In another aspect, the invention features a pharmaceutical composition including one or more siRNA molecules described above and a pharmaceutically acceptable carrier. The pharmaceutical composition can also include, for example, diluents, lubricants, binders, carriers, disintegration means, absorption means, colourings, sweeteners and/or flavourings, adjuvants, and/or other therapeutically principles. In preferred embodiments, the siRNA molecule includes a sequence that is at least 90% homologous, preferably 95%, 99%, or 100% homologous, to any of the sequences set forth in SEQ ID NOs: 1 to 74. Preferably, the pharmaceutical composition includes one or more of the siRNA sequences set forth in SEQ ID NO: 1 (54) or 2 (55). It is also preferred that the pharmaceutical composition includes one or more of the siRNA sequences set forth in SEQ 1N NOs: 9 (62), 10 (63), 11 (64), 24(40), 28(44) or 29(45), more preferably SEQ ID NO: 29 (45). Additional preferred siRNAs are SEQ ID NOs: 33 to 37 (48 to 53), more preferably SEQ ID NOs: 33 (48) or 34 (49).

The invention also features a method to treat, prevent or inhibit unwanted blood coagulation or a coagulation disorder. This method includes administering to a subject a therapeutically effective amount of one or more siRNA molecules described above. In preferred embodiments, the coagulation disorder is selected from the group consisting of coronary artery disease, hypercoagulative disorders, thromboembolic disorders, cardiac ischemia, stroke, myocardial infarction, thrombocytosis, restenosis, and disorders characterized by localized intravascular coagulation. The method can also include administering to the subject a thrombolytic agent. In preferred embodiments, the subject is a vertebrate or a mammal (e.g., a human or a mouse). The siRNA molecule can be single-stranded or double-stranded, preferebaly double-stranded. In additional preferred embodiments, the siRNA molecule includes a sequence that is 90% homologous, more preferably 95%, 99%, or 100% homologous to any of the sequences set forth in SEQ ID NOs: 1 to 74, preferably SEQ ID NOs: 1 to 8 (54 to 61), 32 to 37 (48 to 53), 16 to 23 (69 to 74, 38 to 39), 9 to 21 (62 to 74), 22 to 31 (38 to 47).

The invention also features a method to treat, prevent or inhibit tumor metastasis. This method includes administering to a subject a therapeutically effective amount of one or more siRNA molecules described above. In preferred embodiments, the subject is a vertebrate or a mammal (e.g., a human or a mouse). In additional preferred embodiments, the tumor is selected from the group consisting of bladder, blood, bone, brain, breast, cartilage, colon, kidney, liver, lung, lymph node, nervous tissue, ovarian, pancreatic, prostate, skeletal muscle, skin, spinal cord, spleen, stomach, testicular, thymus, thyroid, trachea, urogenital tract, ureter, urethrea, uterine, and vaginal tumors. The siRNA molecule can be single-stranded or double-stranded, preferably double-stranded. In additional preferred embodiments, the siRNA molecule includes a sequence that is 90% homologous, more preferably 95%, 99%, or 100% homologous to any of the sequences set forth in SEQ ID NOs: 1 to 74, preferably SEQ ID NOs: 1 to 8 (54 to 61), 32 to 37 (48 to 53), 16 to 23 (69 to 74, 38 to 39), 9 to 21 (62 to 74), 22 to 31 (38 to 47).

In yet another aspect, the invention features a method of reducing TF expression levels in a cell. The method includes administering to the cell one or more isolated siRNA molecules described above. In preferred embodiments, the siRNA molecule includes a sequence that is 90% homologous, more preferably 95%, 99%, or 100% homologous to any of the sequences set forth in SEQ ID NOs: 1 to 74, preferably SEQ ID Nos: 1 to 8 (54 to 61), 32 to 37 (48 to 53), 16 to 23 (69 to 74, 38 to 39), 9 to 21 (62 to 74), 22 to 31 (38 to 47). In additional preferred embodiments, the siRNA molecules are double stranded. Desirably, the siRNA molecules are stably expressed in the cell.

The invention also features a kit for the treatment or prevention of a coagulation disorder that includes one or more isolated siRNA molecules described above and instructions for their use in the treatment or prevention of a coagulation disorder. In preferred embodiments, the siRNA molecule includes a sequence that is at least 90% homologous, preferably 95%, 99%, or 100% homologous, to any of the sequences set forth in SEQ ID NOs: 1 to 74.

The invention also features a kit for the treatment or prevention of tumor metastasis that includes one or more isolated siRNA molecules described above and instructions for their use in the treatment or prevention of tumor metastasis. In preferred embodiments, the siRNA molecule includes a sequence that is at least 90% homologous, preferably 95%, 99%, or 100% homologous, to any of the sequences set forth in SEQ ID NOs: 1 to 74.

By “coagulation disorder” is meant any pathological disorder characterized by unwanted blood clot or thrombus formation. Non-limiting examples include coronary artery disease, hypercoagulative disorders, thromboembolic disorders, stroke, myocardial infarction, thrombocytosis, and restenosis. Also included in this definition are disorders characterized by localized intravascular coagulation resulting from the increased expression or coagulation-inducing activity of TF. Non-limiting examples include shock, septicaemia, cardiac arrest, post-operative deep vein thrombosis, pulmonary embolism, unstable angina, post-angioplasty thrombosis, extensive trauma, bites of poisonous snakes, acute liver disease, major surgery, burns, septic abortion, heat stroke, disseminated malignancy, metastasis or tumor-driven angiogenesis, systemic lupus erythematosus, renal disease and eclampsia.

By “double-stranded” is meant a nucleic acid molecule having both a sense and an anti-sense strand. The sense strand and the antisense strand can be from the same nucleic acid molecule or assembled from two nucleic acid molecules and covalently connected via a linker molecule (e.g., a polynucleotide linker or a non-nucleotide linker).

By “homologous” is meant any gene or protein sequence that bears at least 30% homology, more preferably 40%, 50%, 60%, 70%, 80%, and most preferably 90%, 95%, 99%, or 100% homology to a known gene or protein sequence over the length of the comparison sequence. For polypeptides, the length of comparison sequences will generally be at least 16 amino acids, preferably at least 20 amino acids, more preferably at least 25 amino acids, and most preferably 35 amino acids or more. For nucleic acids, the length of comparison sequences will generally be at least 10 nucleotides, preferably at least 15, 16, 17, 18, 19, or 20 nucleotides, more preferably 21, 22, 23, 24, 25, 26, 27, 28, 29, or 30 nucleotides.

By “nucleic acid molecule,” “oligonucleotide,” or “nucleobase oligomer” is meant any chain of nucleotides or nucleic acid mimetics. Included in this definition are natural and non-natural oligonucleotides, both modified and unmodified.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “portion” is meant a fragment of a protein or oligonucleotide that is substantially homologous to a reference protein or oligonucleotide, and retains at least 50% or 75%, more preferably 80%, 90%, or 95%, or even 99% of the biological activity of the reference protein or oligonucleotide using an assay as described herein.

By “positioned for expression” is meant that the oligonucleotide of the invention (e.g., a DNA molecule) is positioned adjacent to a DNA sequence which directs transcription and translation of the sequence (i.e., facilitates the production of, for example, a recombinant polypeptide of the invention, or an RNA molecule).

By “promoter” is meant a polynucleotide sufficient to direct transcription. For example, any polynucleotide region upstream of a gene or a region of an mRNA that is sufficient to direct gene transcription.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “reduce or inhibit” is meant the ability to cause an overall decrease, preferably of 20% or greater, more preferably of 50% or greater, and most preferably of 75% or greater, in the level of protein or oligonucleotide as compared to a reference sample (e.g., a sample not treated with RNAi). This reduction or inhibition of RNA or protein expression can occur through targeted mRNA cleavage or degradation. Assays for protein expression or nucleic acid expression are known in the art and include, for example, ELISA, western blot analysis for protein expression, Southern blotting or PCR for DNA analysis, and northern blotting or RNase protection assays for RNA. By “reduce or inhibit” is also meant an overall decrease preferably of 20% or greater, more preferably of 50% or greater, and most preferably of 75% or greater, in the biological activity of TF. Assays for TF activity are known in the art and include in vitro coagulation assays, one-stage clotting assyas, two-stage clotting assays, TF clotting time assays, and prothrombin time assays.

By “small interfering RNA” or “siRNA” is meant an isolated RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that has been shown to function as a key intermediate in triggering sequence-specific RNA degradation. A range of 19-25 nucleotides is the most preferred size for siRNAs. siRNAs can also include short hairpin RNAs (shRNA) in which both strands of an siRNA duplex are included within a single RNA molecule. Double-stranded siRNAs generally consist of a sense and anti-sense strand and generally include an “i” at the end of the name of the molecule (e.g., hTF167i). Single-stranded siRNAs generally consist of only the antisense strand that is complementary to the target gene. siRNA includes any form of RNA, preferably dsRNA (proteolytically cleaved products of larger dsRNA, partially purified RNA, essentially pure RNA, synthetic RNA, recombinantly produced RNA) as well as modified RNA that differs from naturally occurring RNA by the addition, deletion, substitution, and/or alteration of one or more nucleotides. Such alterations can include the addition of non-nucleotide material, such as to the end(s) of the 19 to 25 nucleotide RNA or internally (at one or more nucleotides of the RNA). In a preferred embodiment, the RNA molecule contains a 3′ hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incoporation) can be found in the published U.S. application publication number 20040019001 (see Summary of the Invention section). Collectively, all such altered RNAs described above are referred to as modified siRNAs.

siRNAs of the present invention can bypass the RNAse III-like RNAi initiator Dicer and directly charge the effector nuclease RISC so that TF mRNA is degraded. siRNAs are synthesized against different parts of TF mRNA, after which they combine with RISC which is then guided for specific degradation/silencing of TF mRNA. siRNAs of the present invention need only be sufficiently similar to natural RNA such that it has the ability to function as a key intermediate in triggering sequence specific RNA degradation, also known as RNAi.

Preferably, RNAi is capable of decreasing the expression of TF in a cell by at least 10%, 20%, 30%, or 40%, more preferably by at least 50%, 60%, or 70%, and most preferably by at least 75%, 80%, 90%, 95% or more.

By “shRNA” is meant an RNA comprising a duplex region complementary to an mRNA, as described in Yu et al. (supra) or Paddison et al. (Proc. Natl. Acad. Sci USA, 99:6047-6052, 2002; Genes & Dev, 16:948-958, 2002). For example, a short hairpin RNA (shRNA) may comprise a duplex region containing nucleotides, where the duplex is between 19 and 29 bases in length, and the strands are separated by a single-stranded 3, 4, 5, 6, 7, 8, 9, or 10 base linker region. Optimally, the linker region is 6 bases in length.

By “subject” is meant a vertebrate, preferably a mammal, including, but not limited to, a human or non-human mammal, such as cow, horse, pig, sheep, mouse, rat, dog, cat, monkey, or baboon.

By “substantially complementary” is meant a nucleic acid sequence that is at least 70%, 80%, 85%, 90% or 95% complementary to at least a portion of a reference nucleic acid sequence. By “complementarity” or “complementary” is meant that a nucleic acid can form hydrogen bond(s) with another nucleic acid sequence by either traditional Watson-Crick or other non-traditional types of interaction. In reference to the nucleic molecules of the present invention, the binding free energy for a nucleic acid molecule with percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “substantially identical” is meant a polypeptide or nucleic acid exhibiting at least 90%, preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, by “substantially identical” is also meant “substantially complementary.” For nucleic acids, the length of comparison sequences will generally be at least 10 nucleotides, preferably at least 14 nucleotides, and more preferably at least 25 nucleotides.

The term “identity” is used herein to describe the relationship of the sequence of a particular nucleic acid molecule or polypeptide to the sequence of a reference molecule of the same type. For example, if a polypeptide or nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment. Methods to determine identity are available in publicly available computer programs. Computer program methods to determine identity between two sequences include, but are not limited to, the GCG program package (Devereux et al., Nucleic Acids Research 12(1): 387, 1984), BLASTP, BLASTN, and FASTA (Altschul et al., J. Mol. Biol. 215: 403 (1990). The well-known Smith-Waterman algorithm may also be used to determine identity. The BLAST and BLAST2 programs are publicly available from NCBI and other sources (BLAST Manual, Altschul, et al., NCBI NLM NIH Bethesda, Md. 20894). Searches can be performed in URLs such as http://www.ncbi.nlm.nih.gov/BLAST or http://www.ncbi.nlm.nih.gov/gorf/b12.html (Tatusova et al., FEMS Microbiol. Lett. 174:247-250, 1999). These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Alternatively, or additionally, two nucleic acid sequences are “substantially identical” if they hybridize under high stringency conditions.

By a “therapeutically effective amount” is meant an amount of a compound, alone or in combination with known therapeutics, that is sufficient to reduce the activity of TF as it relates to coagulation or tumor metastasis. The effective amount of an active compound(s) used to practice the present invention for therapeutic treatment of tumors or coagulation disorders varies depending upon the manner of administration, the age, body weight, and general health of the subject. Ultimately, the attending physician or veterinarian will decide the appropriate amount and dosage regimen. An effective amount of a TF siRNA therapeutic for the treatment of tumors or a coagulation disorder is as little as 0.005, 0.01, 0.025, 0.05, 0.075, 0.1 mg per dose, or as much as 0.15, 0.3, 0.5, 0.6, 0.7, 0.8, 1.0, 1.25, 1.5, 2.0 or 2.5 mg per dose. The dose may be administered once a day, once every two, three, four, seven, fourteen, or twenty-one days. For the treatment of tumors, the amount will be sufficient to effectively reduce or inhibit cell proliferation, tumor size, tumor-driven angiogenesis, or metastasis. For coagulation disorders, the amount will be sufficient to effectively reduce the initiation or progression of the blood clotting cascade that results in clot formation or thrombosis. It will be appreciated that there will be many ways known in the art to determine the therapeutic amount for a given application. For example, the pharmacological methods for dosage determination may be used in the therapeutic context.

By “thrombolytic agent” (also called clotbuster, clot-dissolving medication, fibrinolyic agent) is meant a pharmaceutical compound that is able to dissolve a clot (thrombus) and reopen an artery or vein. Thrombolytic agents are generally serine proteases and convert plasminogen to plasmin which breaks down the fibrinogen and fibrin and dissolves the clot. Currently available thrombolyic agents include reteplase (r-PA or Retavase), alteplase (t-PA or Activase), urokinase (Abbokinase), prourokinase, anisoylated purified streptokinase activator complex (APSAC), and streptokinase.

By “tissue factor protein” is meant any chain of amino acids, regardless of length or post-translational modification (for example, glycosylation or phosphorylation) that is substantially identical to any mammalian TF or TF precursor nucleic acid molecule. See, for example, GenBank accession numbers AAH11029 (human), NP001984 (human), P20352 (mouse), AAH24886 (mouse), AAH16397 (mouse), P42533 (rat), P30931 (bovine), Q9JLU8 (guinea pig). TF is an integral membrane glycoprotein that can trigger blood coagulation via the extrinsic pathway (Bach et al., J. Biol Chem. 256, 8324-8331 (1981)). TF consists of a protein component (previously referred to as TF apoprotein-III) and a phospholipid (Osterud and Rapaport Proc. Natl. Acad. Sci. 74, 5260-5264 (1977)). TF from various organs and species has been reported to have a relative molecular mass of 42,000 to 53,000. Purification of TF has been reported from various tissues such as human brain (Guha et al. Proc. Natl. Acad. Sci. 83, 299-302 (1986) and Broze et al., J. Biol. Chem. 260, 10917-10920 (1985)); bovine brain (Bach et al., J. Biol. Chem. 256, 8324-8331 (1981)); human placenta (Bom et al., Thrombosis Res. 42:635-643 (1986); and, Andoh et al., Thrombosis Res. 43:275-286 (1986)); ovine brain (Carlsen et al., Thromb. Haemostas. 48, 315-319 (1982)); and lung (Glas and Astrup Am. J. Physiol. 219, 1140-1146 (1970)). It has been shown that bovine and human tissue thromboplastin are identical in size and function (see for example Broze et al., J. Biol. Chem. 260, 10917-10920 (1985)). It is widely accepted that while there are differences in structure of TF protein between species, there are no functional differences as measured by in vitro coagulation assays. As used herein, TF includes TF protein from any of the species or tissues described herein having TF biological activity. TF biological activity can be measured by any of several assays known in the art. Non-limiting examples include in vitro coagulation assays, one-stage clotting assays, two-stage clotting assays (Pitlick and Nemerson, Methods Enzymol., 45: 37-48 (1976)), TF clotting time assay (Santucci et al., Thromb. Haemost. 83:445-454, 2000), and prothrombin time assays.

By “tissue factor nucleic acid” is meant a nucleic acid molecule (e.g., DNA, cDNA, genomic, mRNA, RNA, dsRNA, antisense RNA, shRNA) substantially identical to any mammalian sense or antisense TF or TF precursor nucleic acid molecule or any nucleic acid molecule that encodes any of the TF proteins described above. See, for example, GenBank accession numbers M16553 (human), BC011029 (human) NM01993 (human), AF540377 (human), U07619 (rat), M57896 (mouse), and M55390 (rabbit).

By “treating” is meant administering a compound or a pharmaceutical composition for prophylactic and/or therapeutic purposes. To “treat disease” or use for “therapeutic treatment” refers to administering treatment to a subject already suffering from a disease to improve the subject's condition. Preferably, the subject is diagnosed as suffering from a coagulation disorder or a tumor with metastatic potential. To “prevent disease” refers to prophylactic treatment of a subject who is not yet ill, but who is susceptible to, or otherwise at risk of, developing a particular disease. In one example, a subject is determined to be at risk of developing a coagulation disorder based on a family history of coagulation disorders or prior cardiac events. In another example, a subject is deteremined to be at risk of developing a tumor metastasis if the subject has been diagnosed with a malignant tumor. Thus, in the claims and embodiments, treating is the administration to a mammal either for therapeutic or prophylactic purposes.

By “tumor” is meant an abnormal group of cells or tissue that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign or malignant. Non-limiting examples of tumors include bladder, blood, bone, brain, breast, cartilage, colon, kidney, liver, lung, lymph node, nervous tissue, ovarian, pancreatic, prostate, skeletal muscle, skin, spinal cord, spleen, stomach, testicular, thymus, thyroid, trachea, urogenital tract, ureter, urethrea, uterine, and vaginal tumors. By “metastasis” is meant the spead of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential.

Other features and advantages of the invention will be apparent from the following description of the preferred embodiments thereof, and from the claims.

### BRIEF DESCRIPTION OF THE FIGURES

The present invention will now be described in more detail, with reference to figures and examples.

FIGS. 1A-1C show the siRNAs, reporter construct and RNAi of transgene expression. FIG. 1A is a listing of the sense (top) and antisense (bottom) strands of thirteen siRNA species targeting sites within human TF (Genbank entry Acc. No. M16553) mRNA. FIG. 1B is a diagram of the luciferase reporter construct using human TF. FIG. 1C is a graph showing RNAi by siRNA in cotransfection assays (averages of three or more independent experiments each in triplicate, ±s.d. are shown).

FIG. 2 is a graph showing the efficacy of the siRNAs in standard cotransfection assays in HaCaT cells. Different synthetic batches of the hTF167i siRNA showed similar efficacy. The results are averages of at least three experiments, each in triplicate

FIGS. 3A and 3B show siRNA mediated reduction of endogenous TF expression. FIG. 3A is a northern blot showing hTF167i and hTF372i induced cleavage of mRNA in transfected cells. The Northern analysis of TF mRNA was performed after transfection of HaCaT cells with siRNA (100 nM) with GADPH as control. Arrowhead indicates cleavage fragments resulting from siRNA action. FIG. 3B is a graph showing the effect of siRNAs on steady state mRNA levels (filled bars), procoagulant activity (dotted bars), and TF protein (antigen) expression (hatched bars). For measurement of procoagulant activity and antigen, cells were harvested 48 hours after siRNA transfection to accommodate the 7-8 hour half-life of TF protein. Data are from a representative experiment in triplicate.

FIGS. 4A and 4B are graphs showing the time-dependence of siRNA-mediated RNAi. FIG. 4A is a bar graph showing a reduction in inhibitory activity when mutations (M1 and M2 refer to one and two mutations, respectively) are introduced into the siRNAs. Cells were transfected with 100 nM siRNA and harvested for mRNA isolation 4, 8, 24 and 48 hours (filled bars, lined bars, white bars with black dots and hatched bars, respectively). Expression levels were normalised to GADPH and standardised to mock-transfected cells at all time-points. FIG. 4B is a graph showing the time-course of inhibitory effect for mRNA levels (closed diamonds), reporter gene activity (open triangles) and procoagulant activity (filled bars).

FIG. 5 is a graph showing the activity of mutants against endogenous human TF mRNA. HaCaT cells were harvested for mRNA isolation 24 hours post-transfection. TF expression was normalized to that of GAPDH. Normalized expression in mock-transfected cells was set as 100%. Data are averages+s.d. of at least three independent experiments.

FIG. 6 is a graph showing the activity of chemically modified siRNA against endogenous TF mRNA. Experiments were performed and analysed as described in FIG. 5.

FIGS. 7A and 7B are graphs showing the persistence of TF silencing by chemically modified siRNAs. FIG. 7A shows specific TF expression 5 days post-transfection of 100 nM siRNA. FIG. 7B shows a time-course of TF mRNA silencing. Cells were harvested 1, 3, or 5 days after single transfection of 100 nM siRNA. The media was replaced every second day.

FIGS. 8A-8D show the in vitro characterization of mouse TF siRNA activity. FIG. 8A is a Northern blot analysis of B16 cells 24 hours after Lipofectamine2000-mediated transfection with 100 nM of each of eight different siRNA targeting mTF mRNA. Mock-transfected cells were used as control. GAPDH expression served as loading and normalization control. The cleavage fragments of higher mobility than full-length mTF mRNA result from the action of active siRNA. FIG. 8B shows a partial sequence alignment of human and murine TF, comparing the target sequences (underlined) of mTF223i and hTF167i siRNA. FIG. 8C shows a Northern blot analysis of cells transfected with the mismatched siRNA hTF167i, targeting human TF. FIG. 8D is a graph showing the persistence of silencing in B16 cells following transfection of 100 nM mTF223i. Cells were sub-cultured 1 and 3 days after transfection to maintain exponential growth and harvested after 1, 3, 4 and 5 days. Expression of TF mRNA was normalised to GAPDH and standardized to levels in mock-transfected cells.

FIG. 9 shows representative pictures of the lungs of C57 BL/6 mice 10 or 15 days after intravenous injection of TF siRNA-transfected B16 cells. Cells were transfected with siRNA against hTF (hTF167i, control) or either of two different siRNA targeting mTF (mTF223i, mTF321i).

FIGS. 10A and 10B show representative pictures of the lungs from mice injected with hTF167i and mTF223i transfected cells, harvested on day 10 (left panel) and day 15 (right panel). FIG. 10A shows a representative pair of samples from day 10 on the left, together with all day 15 samples from the same experiment. FIG. 10B shows representative pictures of lungs from mice harvested on day 20, in an experiment incorporating three experimental groups (cells treated with hTF167i, mTF223i or mTF321i siRNA).

FIG. 11 is a growth curve showing the effect of siRNA on localized subcutaneous tumor growth. Ten mice in each experimental group were injected subcutaneously with 100 ul 2.5×10⁶/ml B16 cells transfected with either hTF167i or mTF223i siRNA. The growth curves differed significantly on day 8 (P=0.03) and day 9 (P=0.05).

### DETAILED DESCRIPTION OF THE INVENTION

Despite the role of TF in pathologies such as coagulation disorders and tumor metastasis, no clinically useful direct inhibitor of TF has been identified. In addition, there is no evidence that TF can be successfully regulated at the level of gene expression.

The present invention features the discovery of compositions and methods relating to RNA interference that can be used to modulate TF expression. These methods and compositions are useful for the treatment and prevention of coagulation disorders and tumor metastasis.

### RNAi

RNAi is a form of post-transcriptional gene silencing initiated by the introduction of siRNAs. Short 21 to 25 nucleotide double-stranded RNAs are effective at down-regulating gene expression in nematodes (Zamore et al., Cell 101:25-33, 2000) and in mammalian tissue culture cell lines (Elbashir et al., Nature 411:494-498, 2001). The further therapeutic effectiveness of this approach in mammals was demonstrated in vivo by McCaffrey et al. (Nature 418:38-39, 2002). The nucleic acid sequence of a mammalian gene, such as TF, can be used to design small interfering RNAs (siRNAs) that will inactivate TF target genes that have the specific 21 to 25 nucleotide RNA sequences used. siRNAs that target TF may be used, for example, as therapeutics to treat or prevent a coagulation disorder or a metastatic tumor.

Provided with the sequence of a mammalian gene, siRNAs may be designed to inactivate target genes of interest and screened for effective gene silencing, as described herein. General methods for the design of siRNA are disclosed herein. In addition, chemically-synthesized siRNAs can be obtained, for example, from Dharmacon Research Inc. (Lafayette, Colo.), Pharmacia, or ABI. Helpful steps for design and selection are given in Dykxhoorn et al. (Nat. Rev. Mol. Cell. Biol. 4:457-467, 2003), and other approaches are also known in the art (Yu et al., Proc. Natl. Acad. Sci. 99:6047-52, 2002; Sohail et al., Nucleic Acids Res. 31:e38, 2003).

siRNA Design

Methods for producing siRNAs are standard in the art. For example, the siRNA can be chemically synthesized or recombinantly produced. For example, short sense and antisense RNA oligomers can be synthesized and annealed to form double-stranded RNA structures with 2-nucleotide overhangs at each end (Caplen, et al. Proc Natl Acad Sci USA, 98:9742-9747, 2001; Elbashir, et al. EMBO J, 20:6877-88, 2001). These double-stranded siRNA structures can then be directly introduced to cells, either by passive uptake or a delivery system of choice, such as described below.

In some embodiments, siRNAs are generated by processing longer double-stranded RNAs, for example, in the presence of the enzyme dicer under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In other embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides, or pyrimidine nucleotides, such as cytosine, thymine, and uracil, or any combination thereof. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

In some embodiments, the RNAi construct is in the form of a hairpin structure. The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, hairpin RNAs are engineered in cells or in animals to ensure continuous and stable suppression of a desired gene. It is known in the art that siRNAs can be produced by processing a hairpin RNA in the cell.

General guidelines for the requirements and modifications of siRNA are described in PCT publication number WO01/75164. For example, preferred siRNAs may be selected using the following criteria.

Target Sequence GC Ratio

First, preferred siRNAs having 21 or 23 nucleotides are selected in the coding region of an mRNA of interest having a GC ratio close to 50%. Optimally, the GC ratio is between 45% and 55%. Less preferred siRNAs have 60% GC content to 70% GC content. Typically, siRNAs having greater than 70% GC content are not preferred, given that they induce decreased levels of gene silencing relative to siRNAs having preferred levels of GC content.

Target Sequence Position

Second, preferred siRNAs are selected from regions that are not within 50-100 nucleotides of an AUG start codon or within 50-100 nucleotides of the termination codon.

Target Sequence Base Content

Third, preferred siRNAs are selected from target sequences that start with two adenosines. When a target sequence starting with AA is selected, siRNA with dTdT overhangs can be produced. Such siRNAs are easier and less expensive to synthesize, and generally show improved resistance to nucleases. In addition, preferably, the targeted region does not contain three or more consecutive guanosines. Such poly-G sequences can hyperstack and form agglomerates that potentially interfere in the siRNA silencing mechanism.

Target Sequence Specificity

Fourth, preferred siRNAs are selected from target sequences that are not homologous to other genes unrelated to tissue factor. BLAST searches of prospective target sequences are performed to identify those having low homology to nucleic acid sequences other than the gene of interest. This allows the selection of siRNAs having greater specificity and prevents the silencing of genes having homology to the target sequence.

In sum, most preferred target sequences are 21 to 25 nucleotides in length; are within the coding sequence of a gene of interest; start with AA; have 50% GC content; are not within 50-100 nucleotides of a start or termination codon and are not homologous to non-tissue factor genes. When target sequences that meet all of these criteria are used for siRNA target sequence design, RNAi effectively silences more than 80% of target genes. The rate of success can be further improved by selecting at least two target sequences for siRNA design.

### RNAi Target Selection and Identification

While various parameters are used to identify promising RNAi targets, the most effective siRNA and shRNA candidate sequences are identified by empirical testing. One strategy for such testing is to construct a large library of non-overlapping synthetic siRNAs or shRNA encoding vectors that give good coverage of a tissue factor gene of interest, according to its largest sequenced cDNA, which includes partial 5′ and 3′UTR sequences. Provided with knowledge of the intron-exon structure of tissue factor and with sensitive means of measuring target knock-down, such as Taqman quantitative RT-PCR and ELISA assays, the process of siRNA or shRNA selection is relatively straightforward once conditions have been optimized for transfection and target measurements. In addition to the selections methods described herein other selection strategies exist and are described, for example, by Xu et al. Biochem. Biophys. Res. Commun. 306: 712-717, 2003; Zhang et al., Nucleic Acids Res. 31: e72, 2003; and Sommer et al. Oncogene 22, 4266-4280, 2003.

### Modifications

As described herein, the present invention generally features an isolated nucleic acid molecule and includes any and all modification of the nucleic acid molecule. Non-limiting examples of modifications are described below. The term nucleobase oligomer is used below to encompass an isolated nucleic acid molecule that includes any chain of nucleotides or nucleosides, nucleic acid mimetics, and natural or non-natural oligonucleotides.

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

So far, little is known about general effects of mutations or chemical modifications in an siRNA sequence. Traditionally, chemical modification of nucleic acids has inter alia been used to protect single stranded nucleic acid sequences against nuclease degradation and thus extend the half life of the molecule. For example, WO 91/15499 discloses 2′O-alkyl oligonucleotides useful as antisense probes. Also, 2-O-methylation has been used to stabilize hammerhead ribozymes. However, little is known about the effects of chemical modifications of siRNAs.

Specific examples of preferred modifications to the nucleic acid molecules useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified nucleobase oligomers that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050. The relevant methods disclosed in these applications that teach the preparation of the above phosphorus-containing linkages are herein incorporated by reference.

Nucleobase oligomers having modified backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference. The relevant methods disclosed in these applications that teach the preparation of the above oligonucleotides are herein incorporated by reference.

In other oligonucleotides, both the sugar and the internucleoside linkage, i.e., the backbone, are replaced with novel groups. One such oligonucleotide, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O—, S—, or N-alkyl; O—, S—, or N-alkenyl; O—, S— or N—alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920. The relevant methods disclosed in these applications that teach the preparation of such modified sugar structures are herein incorporated by reference.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692. The relevant methods disclosed in these applications that teach the preparation of such modified nucleobases are herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Létt., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941. The relevant methods disclosed in these applications that teach the preparation of such nucleobase oligomer conjugates are herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922. The relevant methods disclosed in these applications that teach the preparation of such hybrid structures are herein incorporated by reference.

### Locked Nucleic Acids

Locked nucleic acids (LNAs) are nucleobase oligomers that can be employed in the present invention. LNAs contain a 2′O, 4′-C methylene bridge that restrict the flexibility of the ribofuranose ring of the nucleotide analog and locks it into the rigid bicyclic N-type conformation. LNAs show improved resistance to certain exo- and endonucleases and activate RNAse H, and can be incorporated into almost any nucleobase oligomer. Moreover, LNA-containing nucleobase oligomers can be prepared using standard phosphoramidite synthesis protocols. Additional details regarding LNAs can be found in PCT publication No. WO 99/14226 and U.S. Patent Application Publication No. US 2002/0094555 A1, each of which is hereby incorporated by reference.

### Arabinonucleic Acids

Arabinonucleic acids (ANAs) can also be employed in methods and reagents of the present invention. ANAs are nucleobase oligomers based on D-arabinose sugars instead of the natural D-2′-deoxyribose sugars. Underivatized ANA analogs have similar binding affinity for RNA as do phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars. The use of ANAs in therapy is discussed, for example, in Damha et al., Nucleosides Nucleotides & Nucleic Acids 20: 429-440, 2001.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Mutations of siRNA

In addition to the modifications described above, mutations of the siRNA molecules directed towards TF are also included in the present invention. Preferred mutations include single base-pair mutations, including but not limited to those described in Example 5 and shown in Table 1, and double base-pair mutations, also including but not limited to those described in Example 5 and shown in Table 1.

Introduction of siRNA into Cells

To simplify the manipulation and handling of the siRNA molecules, the siRNA nucleobase oligomer can be inserted into a cassette where it is operably linked to a promoter. The promoter must be capable of driving expression of the siRNA in the desired target host cell. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum. Gene Ther. 4:151-159, 1993) and mouse mammary tumor virus (MMTV) promoters may also be used. Certain proteins can be expressed using their native promoter. Other elements that can enhance expression can also be included (e.g., enhancers or a system that results in high levels of expression such as a tat gene and tar element). The recombinant vector can be a plasmid vector such as pUC118, pBR322, or other known plasmid vectors, that include, for example, an E. coli origin of replication (see, Sambrook, et al., Molecular Cloning: A Laboratory Manual, Cold Spring Harbor Laboratory press, 1989). The plasmid vector may also include a selectable marker such as the β lactamase gene for ampicillin resistance, provided that the marker polypeptide does not adversely affect the metabolism of the organism being treated. The cassette can also be bound to a nucleic acid binding moiety in a synthetic delivery system, such as the system disclosed in PCT Publication No. WO95/22618.

In one example, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA. PCT application WO01/77350 describes an exemplary vector for bi-directional transcription of a transgene to yield both sense and antisense RNA transcripts of the same transgene in a eukaryotic cell.

The nucleic acid can be introduced into the cells by any means appropriate for the vector employed. Many such methods are well known in the art (Sambrook et al., supra, and Watson et al., “Recombinant DNA”, Chapter 12, 2d edition, Scientific American Books, 1992). Recombinant vectors can be transferred by methods such as calcium phosphate precipitation, electroporation, liposome-mediated transfection, gene gun, microinjection, viral capsid-mediated transfer, polybrene-mediated transfer, or protoplast fusion. For a review of the procedures for liposome preparation, targeting and delivery of contents, see Mannino and Gould-Fogerite, (Bio Techniques, 6:682-690, 1988), Felgner and Holm, (Bethesda Res. Lab. Focus, 11:21, 1989) and Maurer (Bethesda Res. Lab. Focus, 11:25, 1989).

Transfer of the recombinant vector (either plasmid vector or viral vectors) can be accomplished through direct injection into the amniotic fluid or intravenous delivery. Gene delivery using adenoviral vectors or adeno-associated vectors (AAV) can also be used. Adenoviruses are present in a large number of animal species, are not very pathogenic, and can replicate equally well in dividing and quiescent cells. As a general rule, adenoviruses used for gene delivery are lacking one or more genes required for viral replication. Replication-defective recombinant adenoviral vectors can be produced in accordance with art-known techniques (see Quantin et al., Proc. Natl. Acad. Sci. USA, 89:2581-2584, 1992; Stratford-Perricadet et al., J. Clin. Invest., 90:626-630, 1992; and Rosenfeld et al., Cell, 68:143-155, 1992).

For expression of siRNAs or shRNAs within cells, plasmid or viral vectors may contain, for example, a promoter, including, but not limited to the polymerase I, II, and III H1, U6, BL, SMK, 7SK, tRNA polIII, tRNA(met)-derived, and T7 promoters, a cloning site for the stem-looped RNA coding insert, and a 4-5-thymidine transcription termination signal. The Polymerase III promoters generally have well-defined initiation and stop sites and their transcripts lack poly(A) tails. The termination signal for these promoters is defined by the poly-thymidine tract, and the transcript is typically cleaved after the second uridine. Cleavage at this position generates a 3′ UU overhang in the expressed shRNA, which is similar to the 3′ overhangs of synthetic siRNAs.

A variety of methods are available for transfection, or introduction, of dsRNA into mammalian cells. For example, there are several commercially available transfection reagents including but not limited to: TransIT-TKO™ (Minis, Cat. # MIR 2150), Transmessenger™ (Qiagen, Cat. #301525), and Oligofectamine™ (Invitrogen, Cat. # MIR 12252-011). Protocols for each transfection reagent are available from the manufacturer. Additional formulations that aid in the delivery of oligonucleotides or other nucleobase oligomers to cells are described in (see, e.g., U.S. Pat. Nos. 5,656,611, 5,753,613, 5,785,992, 6,120,798, 6,221,959, 6,346,613, and 6,353,055).

The concentration of siRNA used for each target and each cell line varies but in general ranges from 0.05 nM to 500 nM, more preferably 0.1 nM to 100 nM, and most preferably 1 nM to 50 nM. If desired, cells can be transfected multiple times, using multiple siRNAs to optimize the gene-silencing effect.

Stable Expression of siRNA

A DNA template method has been used to create and deliver siRNA molecules (reviewed in T. Tuschl, Nature Biotechnology, 20:446-448, 2002). The siRNA template is cloned into RNA polymerase III transcription units, which normally encode the small nuclear RNA U6 or the human RNAse P RNA H1. These expression cassettes allow for the expression of both sense and anti-sense RNA. The endogenous expression of siRNA from introduced DNA templates is thought to overcome some limitations of exogenous siRNA delivery, in particular the transient loss of phenotype. In fact, stable cell lines have been obtained using these siRNA expression cassettes allowing for a stable loss of function phenotype (Miyagishi M. and Taira K., Nature Biotech., 20:497-500, 2002; Brummelkamp T. R. et al., Science, 296:550-553, 2002). If desired, stable cell lines for RNAi of TF can be generated using the above techniques.

### Assays for Evaluating Gene Silencing Effect

mRNA and protein expression can be analyzed using any of the methods described herein or any of a variety of art known methods including but not limited to northern blot analysis, RNAse protection assays, luciferase or β-gal reporter assays, western blots, and immunological methods such as ELISAs. TF activity can be measured using a one-stage clotting assay as described herein.

### Therapeutic Applications

The siRNAs according to the present invention can be used to down-regulate the expression or biological activity of mammalian TF. Methods for the production and therapeutic administration of siRNAs for in vivo therapies are described in U.S. Patent Application Publications: 20030180756, 2003/0157030, and 20030170891. Methods describing the successful in vivo use of siRNAs are described by Sang et al. (Nature Medicine 9, 347-351 (2003).

Given the role of TF in the initiation of the blood clotting cascade and the potential role for TF in tumor metastasis, the siRNAs of the present invention can be used to treat or prevent coagulation disorders or tumor metastasis in a warm-blooded animal including, but not limited to, a human, cow, horse, pig, sheep, bird, mouse, rat, dog, cat, monkey, baboon, or the like. Treatment generally begins at a hospital so that the doctor can observe the therapy's effects closely and make any adjustments that are needed. The duration of the therapy depends on the coagulation disorder or tumor-metastasis being treated, the age and condition of the patient, the stage and type of the patient's disease, and how the patient's body responds to the treatment. Therapy may be performed at different intervals (e.g., daily, weekly, or monthly). Therapy may be given in on-and-off cycles that include rest periods so that the patient's body has a chance to build healthy new cells and regain its strength.

Therapeutic treatments for metastatic tumors can be used to prevent tumor metastasis, slow the metastasis, slow the tumor-driven angiogenesis, to slow the tumor's growth, to kill or arrest tumor cells that may have spread to other parts of the body from the original tumor, or to relieve symptoms caused by the cancer. Therapeutic treatments for coagulation disorders can be used, for example, to prevent clotting, to dissolve a clot that is already present, or to slow blood clotting. Therapy can also be used as a prophylactic treatment to prevent the future development of blood clots in a patient determined to be at risk.

The compositions according to the present invention may thus comprise one or more of the siRNAs according to the present invention, diluents, lubricants, binders, carriers disintegration and/or absorption means, colourings, sweeteners flavourings etc., all known in the art. Furthermore, the siRNA compositions described herein may also comprise adjuvants and/or other therapeutic principles, and may be administered alone or together with other pharmaceuticals.

A pharmaceutical preparation according to the present invention may be administered e.g. parenterally (e.g., by subcutaneous, intravenous, intramuscular or intraperitoneal injection or infusion of sterile solutions or suspensions), orally (e.g., in the form of capsules, tablets, pills, suspensions or solutions), nasally (e.g., in form of solutions/spray), buccally, rectally (e.g., in the form of suppositories), vaginally (e.g., in the form of suppositories), by inhalation or insufflation (e.g., in the form of an aerosol or solution/spray), via an implanted reservoir, or by any other suitable route of administration, in dosage formulations containing conventional non-toxic pharmaceutically-acceptable carriers, adjuvants and/or vehicles. The pharmaceutical preparation may further be administered in one dose, in divided doses or by way of sustained release devices. Administration may begin before the patient is symptomatic. In one example, intravenous administration can be used to inject siRNAs directly into the blood stream to treat a coagulation disorder. In another example, direct injection of siRNA into tumors can be used to treat metastatic tumors.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for tissue factor modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The siRNA molecules of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756. The relevant methods disclosed in these applications that teach the preparation of such uptake, distribution, and/or absorption are herein incorporated by reference.

The siRNA molecules of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention can be prepared as SATE [(S-acetyl-2-thioethyl)phosphate] derivatives according to the methods disclosed in PCT publication Nos. WO 93/24510 or WO 94/26764.

The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Carbonates or hydrogen carbonates are also possible. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine. The present invention also includes pharmaceutical compositions and formulations that include the siRNA molecules of the invention. The pharmaceutical compositions of the present invention may be administered in a number of ways depending upon whether local or systemic treatment is desired and upon the area to be treated.

The formulations can be administered to human patients in therapeutically effective amounts (e.g., amounts which prevent, eliminate, or reduce a pathological condition) to provide therapy for a disease or condition. The preferred dosage of an siRNA molecule of the invention is likely to depend on such variables as the type and extent of the disorder, the overall health status of the particular patient, the formulation of the compound excipients, and its route of administration.

In providing a mammal with the siRNA molecules of the present invention the dosage of administered siRNAs will vary depending upon such factors as the mammal's age, weight, height, sex, general medical condition, previous medical history, disease progression, tumor burden, and the like. The dose is administered as indicated. Other therapeutic drugs may be administered in conjunction with the siRNA molecules. The pharmaceutical composition used for the treatment of tumors may optionally contain other chemotherapeutic agents, antibodies, antivirals, exogenous immunomodulators or the like. The pharmaceutical composition used for the treatment of coagulation disorders may optionally contain additional thrombolytic agents or anticoagulants such as heparin.

The efficacy of treatment using the siRNAs described herein may be assessed by determination of alterations in the expression, concentration, or biological activity of the DNA, RNA or gene product of TF; clot dissolution; clot prevention; tumor regression; metastasis regression; metastasis prevention; or a reduction of the pathology or symptoms associated with the tumor.

### EXAMPLES

The invention will now be described by way of examples. Although the examples represent preferred embodiments of the present inventions, they are not to be contemplated as restrictive to the scope of the present invention.

The experiments described below demonstrate that in mammalian cells double-stranded siRNA synthesized to be complementary to a certain partial sequence on the targeted TF mRNA sequence induces degradation of this specific mRNA (see Example 1). This effect was highly sequence-dependent, and contrary to data in lower organisms, as only a few sites on the TF mRNA were highly susceptible to the corresponding siRNAs. As can be seen from Example 2, the depletion of TF mRNA results in marked reduction of TF protein and procoagulant activity, which, according to the knowledge of the present inventors, is the first demonstration of such a reproducible effect on TF not accompanied by toxic side effects to the cell. Based on these results, two positions of susceptibility against TF mRNA are provided.

We demonstrate that a wide range of mutational and chemical modifications of our best siRNA candidate, hTF167i, are well tolerated. The chemical modifications did show some loss of activity with allyl-modifications to the 5′ end, and some expected toxicity with longer stretches thio-phosphates, but the siRNA with 2′-OH-methylated ends both show strong activity and increased life-span in time-course experiments.

### Methods

The following materials and methods were used in the experiments described below.

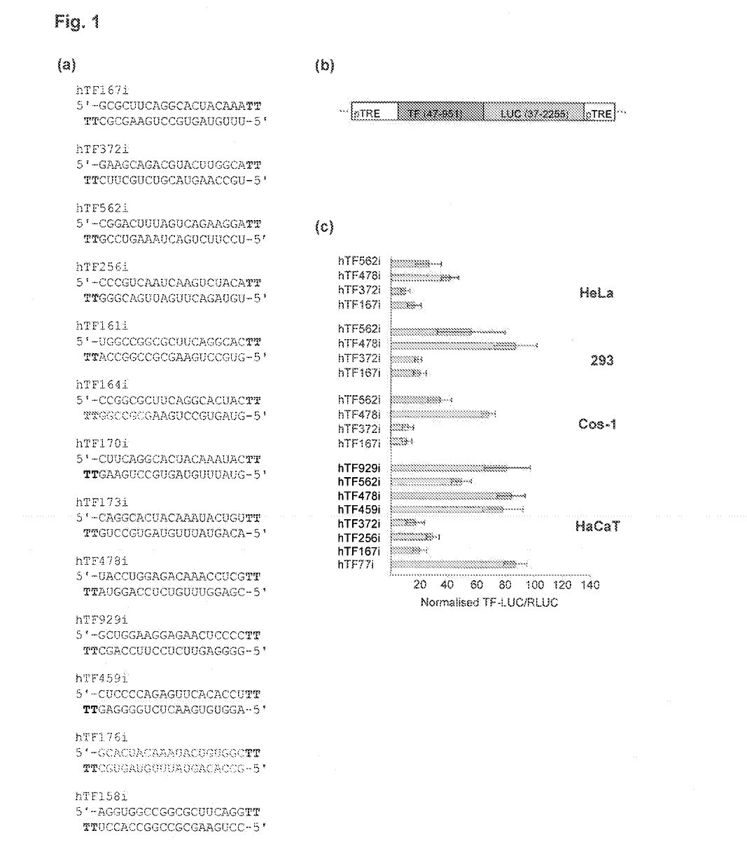

siRNAs targeting human TF (hTF) were designed as follows: 21-nucleotide RNAs were chemically synthesized using phoshoramidites (Pharmacia and ABI). Deprotected and desilylated synthetic oligoribonucleotides were purified on reverse phase HPLC. Ribonucleotides were annealed at 10 μM in 500 μl 10 mM Tris-HCl pH 7.5 by boiling and gradual cooling in a water bath. Successful annealing was confirmed by non-denaturing polyacrylamide gel electrophoresis. siRNA species were designed each targeting a site within human TF mRNA (GenBank Accession No. M16553). The siRNA species were designed with 2 nucleotides deoxythymidine 3′ overhangs and named according to the position of the first nucleotide of the sense strand, using the numbering of the above Genbank entry (FIG. 1A). The present invention includes the synthesised siRNAs set forth in SEQ ID NO: 1 to SEQ ID NO: 74 (Table 1).

TABLE 1
Exemplary siRNAs

| SEQ ID | siRNA sequence | name | strand |
|---|---|---|---|
| 1 | gcgcuucagg cacuacaaan n | hTF167s | sense |
| 2 | gaagcagacg uacuuggcan n | hTF372s | sense |
| 3 | cggacuuuag ucagaaggan n | hTF562s | sense |
| 4 | cccgucaauc aagucuacan n | hTF256s | sense |
| 5 | uggccggcgc uucaggcacn n | hTF161s | sense |
| 6 | ccggcgcuuc aggcacuacn n | hTF164s | sense |
| 7 | cuucaggcac uacaaauacn n | hTF170s | sense |
| 8 | caggcacuac aaauacugun n | hTF173s | sense |
| 9 | ccgcuucagg cacuacaaau a | 167s-s1 | sense |
| 10 | gggcuucagg cacuacaaau a | 167s-s2 | sense |
| 11 | gcccuucagg cacuacaaau a | 167s-s3 | sense |
| 12 | gcgguucagg cacuacaaau a | 167s-s4 | sense |
| 13 | gcgcuugagg cacuacaaau a | 167s-s7 | sense |
| 14 | gcgcuucagc cacuacaaau a | 167s-s10 | sense |
| 15 | gcgcuucagg gacuacaaau a | 167s-s11 | sense |
| 16 | gcgcuucagg caguacaaau a | 167s-s13 | sense |
| 17 | gcgcuucagg cacuagaaau a | 167s-s16 | sense |
| 18 | gcgcuugagc cacuacaaau a | 167s-ds7, 10 | sense |
| 19 | gcgcuucagc gacuacaaau a | 167s-ds10, 11 | sense |
| 20 | gcgcuucagc caguacaaau a | 167s-ds10, 13 | sense |
| 21 | gcgcuucagc cacuagaaau a | 167s-ds10, 16 | sense |
| 22 | g*cgcuucagg cacuacaaau*a | 167s-P1 + 1 | sense |
| 23 | gcgcuucagg cacuacaaa*u*a | 167s-P0 + 2 | sense |
| 24 | g*c*gcuucagg cacuacaaa*u*a | 167s-P2 + 2 | sense |
| 25 | g*c*gcuucagg cacuaca*a*a*u*a | 167s-P2 + 4 | sense |
| 26 | Gcgcuucagg cacuacaaau A | 167s-M1 + 1 | sense |
| 27 | gcgcuucagg cacuacaaaU A | 167s-M0 + 2 | sense |
| 28 | GCgcuucagg cacuacaaaU A | 167s-M2 + 2 | sense |
| 29 | GCgcuucagg cacuacaAAU A | 167s-M2 + 4 | sense |
| 30 | Gcgcuucagg cacuacaaau A | 167s-A1 + 1 | sense |
| 31 | gcgcuucagg cacuacaaaU A | 167s-A0 + 2 | sense |
| 32 | caggcauucc agagaaagcg u | mTF220s | sense |
| 33 | gcauuccaga gaaagcguuu a | mTF223s | sense |
| 34 | cagauaagug aucgaucuag a | mTF321s | sense |
| 35 | agugcuucuc gaccacagac a | mTF355s | sense |
| 36 | uuccagagaa agcguuuaau u | mTF226s | sense |
| 37 | cagagaaagc guuuaauuua a | mTF229s | sense |
| 38 | u*uuguagugc cugaagcgcc*g | 167as-P1 + 1 | antisense |
| 39 | uuuguagugc cugaagcgc*c*g | 167as-P0 + 2 | antisense |
| 40 | u*u*uguagugc cugaagcgc*c*g | 167as-P2 + 2 | antisense |
| 41 | u*u*uguagugc cugaagc*g*c*c*g | 167as-P2 + 4 | antisense |
| 42 | Uuuguagugc cugaagcgcc G | 167as-M1 + 1 | antisense |
| 43 | uuuguagugc cugaagcgcC G | 167as-M0 + 2 | antisense |
| 44 | UUuguagugc cugaagcgcC G | 167as-M2 + 2 | antisense |
| 45 | UUuguagugc cugaagcGCC G | 167as-M2 + 4 | antisense |
| 46 | Uuuguagugc cugaagcgcc G | 167as-A1 + 1 | antisense |
| 47 | uuuguagugc cugaagcgcC G | 167as-A0 + 2 | antisense |
| 48 | gcuuucucug gaaugccugc a | mTF220as | antisense |
| 49 | aacgcuuucu cuggaaugcc u | mTF223as | antisense |
| 50 | uagaucguac acuuaucugu a | mTF321as | antisense |
| 51 | ucuguggucg agaagcacuu g | mTF355as | antisense |
| 52 | uuaaacgcuu ucucuggaau g | mTF226as | antisense |
| 53 | aaauuaaacg cuuucucugg a | mTF229as | antisense |
| 54 | uuuguagugc cugaagcgcn n | hTF167as | antisense |
| 55 | ugccaaguac gucugcuucn n | hTF372as | antisense |
| 56 | uccuucugac uaaaguccgn n | hTF562as | antisense |
| 57 | uguagacuug auugacgggn n | hTF256as | antisense |
| 58 | gugccugaag cgccggccan n | hTF161as | antisense |
| 59 | guagugccug aagcgccggn n | hTF164as | antisense |
| 60 | guauuuguag ugccugaagn n | hTF170as | antisense |
| 61 | acauuuguag ugccugaagn n | hTF173as | antisense |
| 62 | uuuguagugc cugaagcggc c | 167as-s1 | antisense |
| 63 | uuuguagugc cugaagcccc g | 167as-s2 | antisense |
| 64 | uuuguagugc cugaagggcc g | 167as-s3 | antisense |
| 65 | uuuguagugc cugaaccgcc g | 167as-s4 | antisense |
| 66 | uuuguagugc cucaagcgcc g | 167as-s7 | antisense |
| 67 | uuuguagugg cugaagcgcc g | 167as-s10 | antisense |
| 68 | uuuguagucc cugaagcgcc g | 167as-s11 | antisense |
| 69 | uuuguacugc cugaagcgcc g | 167as-s13 | antisense |
| 70 | uuucuagugc cugaagcgcc g | 167as-s16 | antisense |
| 71 | uuuguagugg cucaagcgcc g | 167as-ds7, 10 | antisense |
| 72 | uuuguagucg cugaagcgcc g | 167as-ds10, 11 | antisense |
| 73 | uuuguacugg cugaagcgcc g | 167as-ds10, 13 | antisense |
| 74 | uuucuagugg cugaagcgcc g | 167as-ds10, 16 | antisense |

Notes:
n = any base
*: phosphorothioate linkage
upper case letters: 2-O-methyl nucl.
underlined upper case: 2-O-allyl nucl.

In order to determine whether mutations were equally tolerated within the whole siRNA, the siRNAs according to the present invention were mapped more systematically. To avoid affecting the duplex stability of the siRNA, only GC pairs were targeted for mutation by inversion of the pairs as described in Example 5, below. In addition, given the transient nature of the effect of siRNAs in human cells, chemical modifications were introduced in both ends of the siRNA in an attempt to increase the intracellular stability of siRNA without compromising activity.

The reporter constructs of human TF to be used in the Dual Luciferase system (Promega) were designed using the coding region of TF which were cloned in-frame with the Firefly luciferase (LUC) gene, producing the fusion construct TF-LUC (Acc. No. AF416989). Numbering of the fusion construct refers to that of the GenBank entry for TF (M16553) and to the pGL3-enhancer plasmid (Promega) for LUC. The plasmid pcDNA3-Rluc (Acc. No. AF416990), encoding Renilla luciferase (Rluc) was used as internal control. Regions of TF and LUC cDNA contained within the construct are indicated in FIG. 1B. The dual luciferase system used herein provides a measure of how much TF mRNA is degraded by siRNA(s).

HeLa, Cos-1 and 293 cells were maintained in Dulbecco's Minimal Essential Medium (DMEM) supplemented with 10% fetal calf serum (Gibco BRL). The human keratinocyte cell line HaCaT was cultured in serum free keratinocyte medium supplemented with 2.5 ng/ml epidermal growth factor and 25 μg/ml bovine pituitary extract. All cell lines were regularly passaged at sub-confluence. The day before the experiment cells cultured in DMEM were trypsinized and resuspended in full medium before plating. HaCaT cells were trypsinized until detachment. Trypsin inhibitor was then added and the cells centrifuged for 5 minutes at 400×g before resuspension in supplemented medium and plating. Cells were transfected one or two days later.

Lipofectamine-mediated transient co-transfections were performed in triplicate in 12-well plates with 0.40 μg/ml plasmid (0.38 μg/ml reporter and 20 ng/ml control) and typically 30 nM siRNA (0.43 μg/ml). Luciferase activity levels were measured on 25 μl cell lysate 24 hours after transfection using the Dual Luciferase assay (Promega). Serial transfections were performed by transfecting initially with 100 nM siRNA, followed by transfection with reporter and internal control plasmids before harvest time points.

For northern analyses, HaCaT cells in 6-well plates were transfected with 100 nM siRNA in serum-free medium. Lipofectamine2000™ was used for higher transfection efficiency. Poly(A) mRNA was isolated 24 hours after transfection using Dynabeads oligo(dT)₂₅ (Dynal). Isolated mRNA was fractionated for 16-18 hours on 1.3% agarose/formaldehyde (0.8 M) gels and blotted on to nylon membranes (MagnaCharge, Micron Separations Inc.). Membranes were hybridised with random-primed TF (position 61-1217 in cDNA) and GAPDH (1.2 kb) cDNA probes in PerfectHyb hybridisation buffer (Sigma) as recommended by the manufacturer.

For TF activity measurements HaCaT cell monolayers were washed thrice with ice-cold barbital buffered saline (BBS) pH 7.4 (BBS, 3 mM sodium barbital, 140 mM NaCl) and scraped into BBS. Immediately after harvesting and homogenisation the activity was measured in a one-stage clotting assay using normal citrated platelet poor plasma mixed from two donors and 10 mM CaCl₂. The activity was compared to a standard. One unit (U) TF corresponds to 1.5 ng TF as determined in the TF ELISA. The activity was normalised to the protein content in the cell homogenates, as measured by the BioRad DC assay.

TF antigen was quantified using the Imubind TF ELISA kit (American Diagnostica, Greenwich, Conn.). This ELISA recognises TF apoprotein, TF and TF:Coagulation Factor VII (FVII) complexes. The samples were left to thaw at 37° C. and homogenised. An aliquot of each homogenate (100 μl) was diluted in phosphate-buffered saline containing 1% BSA and 0.1% Triton X-100. This sample was then added to the ELISA-well according to the manufacturer's protocols. The antigen levels were normalized to the total protein content in the cell homogenates.

All the various mutant siRNAs were analysed for depletion of endogenous TF mRNA in HaCaT cells, 24 hours after LIPOFECTAMINE 2000-mediated transfection, as described above.

### Example 1

### Analysis of RNAi in Cells Transiently Cotransfected with hTF-LUC and hTF siRNA

The initial analysis of TF siRNA efficacy was performed in HeLa cells transiently cotransfected with hTF-LUC (FIG. 1B) and hTF siRNA (FIG. 1A) using the Dual Luciferase system (Promega). Ratios of LUC to Rluc expression were normalised to levels in cells transfected with a representative irrelevant siRNA, Protein Serine Kinase 314i (PSK314i).

The siRNAs had potent and specific effects in the cotransfection assays, with the best candidates, hTF167i and hTF372i, resulting in only 10-15% residual luciferase activity in HeLa cells (FIG. 1C). Furthermore, also a positional effect was found, as hTF562i showed only intermediate effect, and hTF478i had very low activity. This pattern was also found in 293, COS-1 and HaCaT cells (FIG. 1C), and with siRNAs from different synthetic batches and at various concentrations. The siRNAs caused the same degree of inhibition over a concentration range of 1-100 nM in cotransfection assays.

Co-culturing siRNA transfected cells with reporter plasmid transfected cells, both in HeLa cells and in the contact-inhibited growth of HaCaT cells, gave no indication of siRNA transfer between cells.

### Example 2

### Investigation of siRNA Position-Dependence

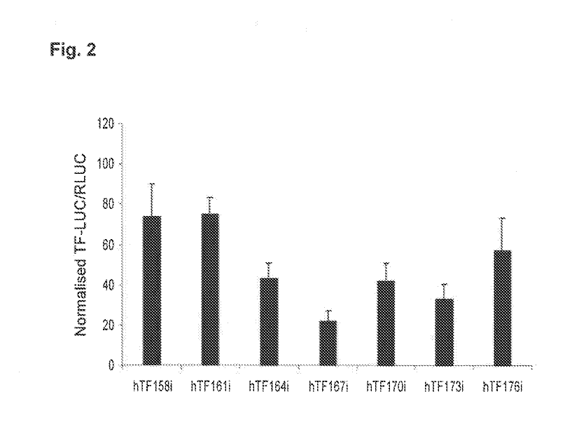

The accessibility of the region surrounding the target site of the preferred siRNA (i.e., hTF167i) at a higher resolution was investigated. siRNAs (hTF158i, hTF161i, hTF164i, hTF170i, hTF173i and hTF176i) were synthesized to target sites shifted at both sides of hTF167i in increments of 3 nucleotides, wherein each of them shared 18 out of 21 nucleotides with its neighbours (FIG. 1C). It was found that despite the minimal sequence and position-differences between these siRNAs, they displayed a wide range of activities (FIG. 2). There was a gradual change away from the full activity of hTF167i that was more pronounced for the upstream siRNAs. The two siRNAs hTF158i and hTF161i were shifted only nine and six nucleotides away, respectively, from hTF167i, yet their activity was severely diminished. These results suggest that local factor(s) caused the positional effect.

### Example 3

### Analysis of hTF siRNA Efficacy on Endogenous mRNA

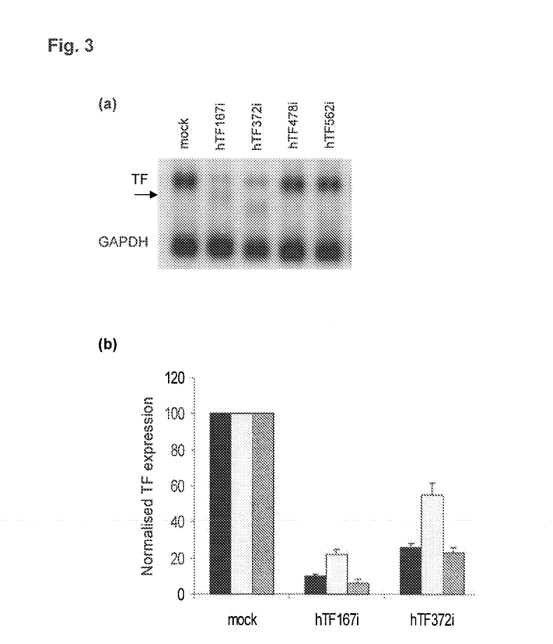

The results of cotransfection assays involving the use of forced expression of reporter genes as substrates may be difficult to interpret. The effect of siRNA was therefore also measured on endogenous mRNA targets in HaCaT cells (FIG. 3A) which express TF constitutively. The two best TF siRNAs, hTF167i and hTF372i, showed strong activity in this assay, as normalised TF mRNA was reduced to 10% and 26%, respectively (FIG. 3A). Cleavage products, whose sizes were consistent with primary cleavages at the target sequences, were clearly visible below the depleted main band. Thus, the present invention also relates to siRNAs that can cleave mRNA in mammalian cells and suggests that RISC may be active also in mammals. The third best siRNA in cotransfection assays, hTF256i, also resulted in significant depletion of TF mRNA levels (57% residual expression). The remaining TF siRNAs did not show any activity as measured by northern assays (FIG. 3B), nor did they stimulate TF expression, a point of some interest, as transfection with chemically modified ribozymes can induce TF mRNA three-fold. Thus, this relative inertness of irrelevant siRNAs (i.e., siRNAs with non-specific effects) further enhances the promise of siRNA-based drugs.

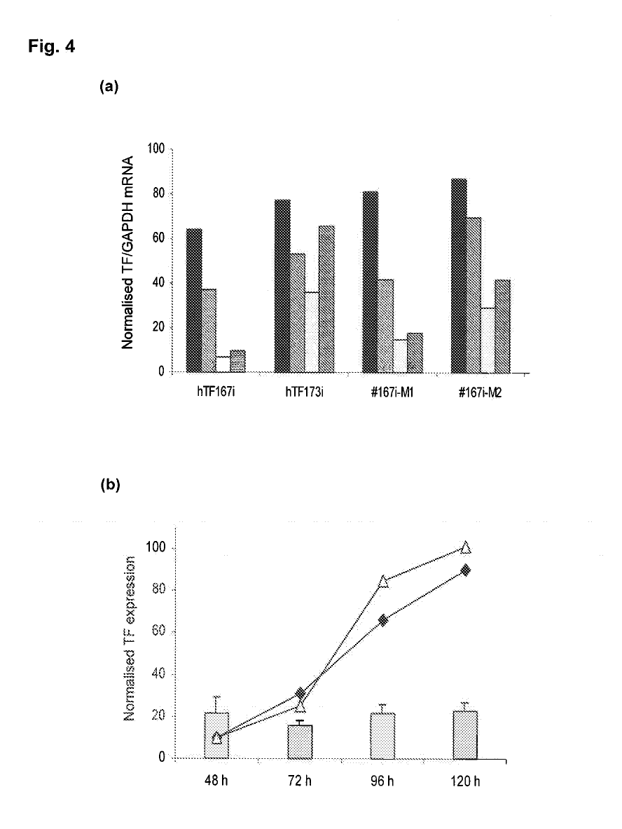

The coagulation activity in the HaCaT cells was reduced 5-fold and 2-fold, respectively, in cells transfected with hTF167i and hTF372i, compared to mock-transfected cells (FIG. 3B and FIG. 4B). The effect of siRNAs on total cellular TF protein was also measured (FIG. 3B), and demonstrated an inhibitory effect that was generally greater than the observed effect on procoagulation activity. Thus, according to the present invention, we conclude that the siRNAs hTF167i and hTF372i display specificity and potency in a complex physiological system, and are not affected by positional effects, as other siRNA molecules against the same target mRNA are basically inactive. The inactivity of certain siRNAs might be due to mRNA folding structure or blockage of cleavage sites by impenetrable protein coverage.

### Example 4

### Analysis of the Time-Course and Persistence of siRNA Silencing

The time-course of mRNA silencing was measured by northern analysis of cells harvested 4, 8, 24 and 48 hours after start of transfection. A maximum siRNA silencing effect was seen after 24 hours (FIG. 4A). There seemed to be a difference in the apparent depletion rate, as hTF167i reduced the mRNA level more than hTF173i at each time-point. Similar observations were made for modified versions of hTF 167i, in which the induced mutations (M1 and M2) resulted in reduced inhibitory activity. Mutations in the anti-sense strand had a more pronounced effect than the corresponding mutations in the sense strand. The fact that siRNA-induced target degradation was incomplete (a level of approximately 10% of the target mRNA remained even with the most effective siRNAs), may be due to the presence of a fraction of mRNA in a protected compartment, such as spliceosomes or in other nuclear locations. However, in view of the above data and data from competition experiments, a more likely possibility may be a kinetically determined balance between transcription and degradation, the latter being a time-consuming process.

Experiments in plants and nematodes have suggested the existence of a system whereby certain siRNA genes are involved in the heritability of induced phenotypes. To investigate the existence of such propagators in mammalian cell lines, the persistence of the siRNA silencing in HaCaT cells transfected at a very low cell density was measured. In an experiment based on serial transfection of reporter constructs there was a gradual recovery of expression between 3 and 5 days post-transfection, and the time-dependence of the siRNA effect on endogenous TF mRNA was similar (FIG. 4B). The level of TF mRNA in mock-transfected control cells declined gradually during the experiment, in what appeared to be cell expansion-dependent down-regulation of expression. The procoagulant activity showed little indication of recovering to control levels in transfected cells (FIG. 4B, columns). Similar observations were made with hTF372i and with a combination of hTF167i, hTF372i and hTF562i.

### Example 5

### Analysis of the Effect of Introducing Base-Pairing Mutations in the siRNA Sequences

The siRNA were mapped more systematically in order to determine whether mutations were equally tolerated within the whole siRNA. A total of eight different single-mutant siRNA were designed and named according to the position (starting from the 5′ of the sense strand) of the mutation (s1, s2, s3, s4, s7, s11, s13, s16). The previously described central single-mutant M1 (Example 4) was included in this analysis and renamed s10. All siRNAs were analysed for productive annealing by denaturing PAGE (15%).

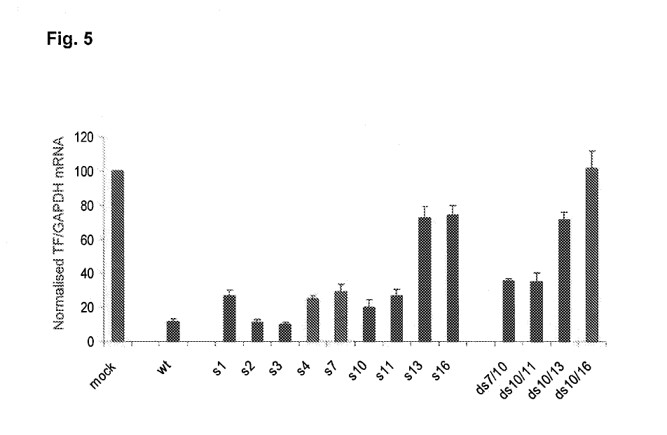

All the various mutant siRNAs were analysed for depletion of endogenous TF mRNA in HaCaT cells, 24 hours after LIPOFECTAMINE2000-mediated transfection, as previously described. A summary of the data is shown in FIG. 5. The wild-type siRNA, and the mutant s10, included as positive controls, depleted TF mRNA to approximately 10% and 20% residual levels, as expected and previously reported. The activities of the other mutants fall in three different groups depending on their position along the siRNA. Mutations in the extreme 5′ end of the siRNA (s1-s3) were very well tolerated, exhibiting essentially the same activity as the wild type. Mutations located further in, up to the approximate midpoint of the siRNA (s4, s7, s10, s11), were slightly impaired in their activity, resulting in depletion of mRNA to 25-30% residual levels. Both the mutations in the 3′ half of the siRNA, however, exhibited severely impaired activity. This suggested to us a bias in the tolerance for mutations in the siRNA. The activities of several double mutants, in which the central position (s10) was mutated in conjunction with one additional position (s7, s11, s13, s16), were also analysed. The bias in mutation tolerance was also evident for these double mutants, as the rank order of their activity mirrored that of the activity of the single mutants of the variant position. This observation strengthens the proposition that the differential activity of mutants is due to an intrinsic bias in the tolerance for target mismatches along the sequence of the siRNA. The reason for such a bias might be linked to the proposed existence of a ruler region in the siRNA which is primarily used by the RISC complex to “measure up” the target mRNA for cleavage.

### Example 6

### Effects of Chemical Modification of the siRNA Sequences

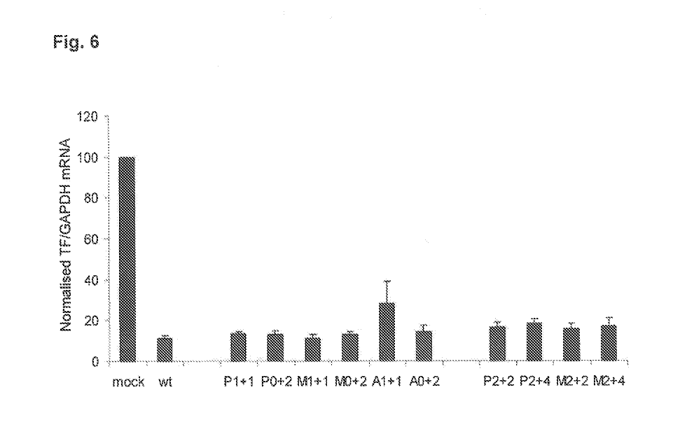

A series of siRNAs with one modification each in the extreme 5′ and 3′ ends of the siRNA strands (P1+1, M1+1, A1+1, i.e. SEQ ID NO 22(38), 26(42) and 30(46), respectively, where the number in parentheses indicates the SEQ ID NO of the complementary sequence) was initially synthesized. The 5′ end of the chemically synthesized siRNAs might be more sensitive to modification since it has to be phosphorylated in vivo to be active. We therefore also included siRNAs with two modifications only in the non-base pairing 3′ overhangs (siRNAs P0+2, M0+2 and A0+2, i.e., SEQ ID NOs: 23(39), 27(43) and 31(47), respectively, Table 1), which were known to be tolerant for various types of modifications. Northern analysis of transfected HaCaT cells demonstrated essentially undiminished activity of all the modified siRNAs, with the exception of the siRNA with allylation at both ends (FIG. 6). Allyl-modification in the 3′ end only had no effect on activity. The presence of a large substituent in 2′-hydroxyl of 5′ terminal nucleotide might interfere with the proper phosphorylation of the siRNA shown to be necessary by Nykänen et al, Cell 107:309-321, 2001).

We next determined if any of these mutations were sufficient to increase the persistence of siRNA-mediated silencing. Endogenous TF mRNA recovers gradually 3-5 days after transfection with wild type siRNA targeting hTF167. No difference was detected in HaCaT cells transfected with active and chemically modified siRNA in parallel 3 and 5 days post-transfection. The moderate modifications we had introduced, although exhibiting full initial activity, were therefore clearly not sufficient to substantially stabilize the siRNAs in vivo.

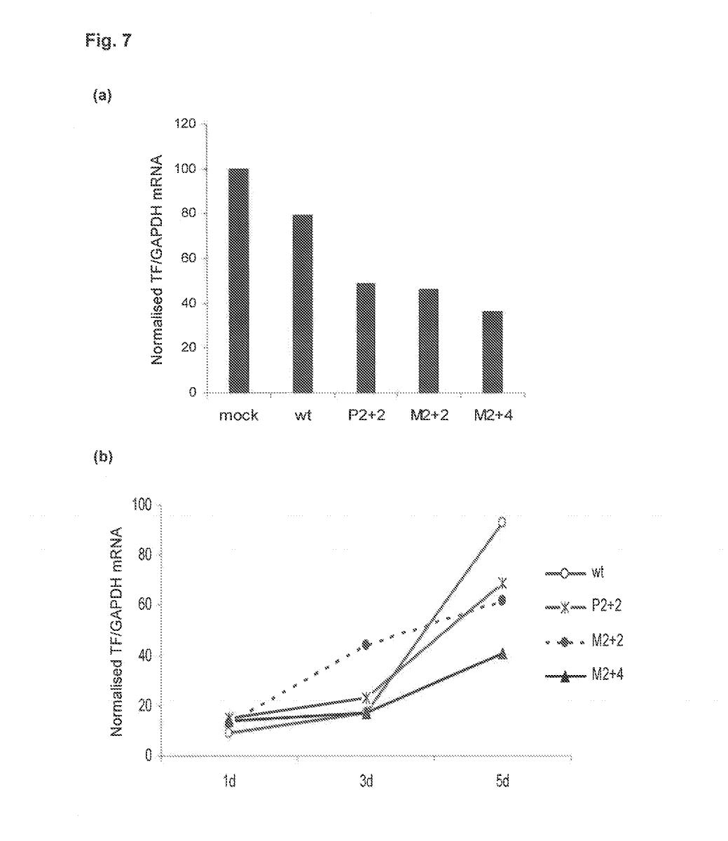

With the activity of the siRNA still intact after our initial moderate modifications, the degree of modifications was extended to include either two on both sides or two on the 5′ end in combination with four in the 3′ end. The new set of siRNAs were analysed for initial activity 24 hours following transfection into HaCaT cells. Normalized expression levels in cells transfected with modified siRNAs were slightly elevated, at 16-18% residual levels, compared to 11% in cells transfected with wild type. The most extensively phosphorothioated siRNA proved to be toxic to cells, resulting in approximately 70% cell death compared to mock-transfected cells (measured as the expression level of the control mRNA GAPDH). Due to these complications, this siRNA species was not included in further analysis. The remaining siRNA species were evaluated for increased persistence of silencing by analysing TF mRNA expression 5 days after a single transfection of 100 nM siRNA. At this point, TF expression in cells transfected with wild-type siRNA had recovered almost completely (80% residual expression compared to mock-transfected cells) (FIG. 7A). In cells transfected with the most extensively modified siRNA (M2+4; SEQ ID NO: 29(45)), however, strong silencing was still evident (32% residual expression). The less extensively modified siRNA species (P2+2, M2+2; SEQ ID NO: 24(40) and SEQ ID NO: 28(44) respectively), although less effective than Me2+4, consistently resulted in lower TF expression 5 days post-transfection (55-60%) than the wild type. Time-course experiments demonstrated that the wild type siRNA was still the most effective 3 days post-transfection, when silencing was relatively unimpaired, but that silencing drops off at a much higher rate thereafter (FIG. 7B).

### Example 7

### siRNA Toward Murine TF Reduces Circulating Malignant Cells Ability to Form Pulmonary Tumors

The experiments were designed and carried out to investigate if knockdown of TF reduced the ability of tail vein-injected cells to settle in the pulmonary circulation and form lung tumors in mice. The cells were transfected with siRNA against murine TF before injection of the knockdown cells, which had a reduction in their TF level down to 10-20% of control mouse before injection. The mice were sacrificed 6-25 days after injection of the cells. In each mouse had 0.2 to 1.0 million cells in 0.2 ml medium been injected. All mice used were C57B1. Three groups were tested in each experiment. Group 1: pretreatment of cells before injection was with siRNA against PSKH1, a serine kinase of unknown function. Group 2: pretreatment of the cells with siRNA against murine TF (mTF223, SEQ ID NO: 33). Group 3: pretreatment of the cells with siRNA against human TF (hTF167, SEQ ID NO: 1). The number of macroscopically visible tumors in the lungs was counted after autopsy of the mice. Examples of the results are given in Tables 2 and 3. Mice receiving cells pretreated with siRNA against murine TF (Group 2) develop a low number of tumors. Group 1 mice develop around 10 times more tumors than Group 2, and mice of Group 3 have more than 500 tumors in their lungs. Since the effect of siRNA on its target mRNA is highly specific (if the siRNA sequence has been properly selected to not bind other nucleotide sequences), the Group 3 mice may have up to 200-250-fold increase in their lung tumors when compared to mice in Group 2. The experiments demonstrate that the level of TF in the injected cells shows a highly specific effect on the ability to form tumors in the lungs.

These results demonstrate that siRNA compositions directed towards TF are useful in the prevention of metastasis and may be used in the treatment of cancer in mammals. Additional specific TF siRNA molecules adapted to the TF sequence of a specific species may be found by the screening methods as described in Holen et al. Nuc. Acids Res. 30:1757-1766, 2002, Amarzguioui et al., Nuc. Acids Res. 31:589-595, 2003, and Holen et al., Nuc. Acids Res. 31:2401-2407, 2003.

TABLE 2
Number of pulmonary metastases 10 days after injection
of TF siRNA-transfected B16 cells in C57 mice.

| | Mouse | Mouse | Mouse | Mouse | Mouse | |
|---|---|---|---|---|---|---|
| Group | 1 | 2 | 3 | 4 | 5 | Mean |
| 1 | 82 | 11 | 5 | 79 | 36 | 43 |
| 2 | 12 | 0 | 37 | 64 | 4 | 23 |
| 3 | 188 | 127 | 13 | 404 | 263 | 199 |

TABLE 3
Number of pulmonary metastases 15 days after injection
of TF siRNA-transfected B16 cells in C57 mice.

| | Mouse | Mouse | Mouse | Mouse | Mouse | Mouse | |
|---|---|---|---|---|---|---|---|
| Group | 1 | 2 | 3 | 4 | 5 | 6 | Mean |
| 1 | 239 | 174 | 56 | 79 | 66 | 342 | 159 |
| 2 | 17 | 12 | 2 | 13 | 11 | 10 | 11 |
| 3 | >500 | >500 | >500 | >500 | >500 | >500 | >500 |
| | Con- | Con- | Con- | Con- | Con- | Con- | Con- |
| | fluent | fluent | fluent | fluent | fluent | fluent | fluent |

### Example 8

### Effect of Mouse siRNA on Mouse TF

In order to determine the effect of targeting mouse TF using mouse specific siRNA, we designed siRNAs against eight different sites within the coding region of murine Tissue Factor targeting sites located within 200 bp corresponding to the region harboring the best siRNA targets in human TF (mTF217i, mTF220i, mTF223i, mTF245i, mTF269i, mTF321i, mTF355i, mTF395i). The siRNAs are named according to the position of the 5′ nucleotide in the duplex target sites within mTF mRNA (Acc. M26071). hTF167i, targeting human TF, has no activity against mTF mRNA and was used as control.

B16 cells were transfected as follows. B16-F10 cells (ATCC: CRL-6475) were maintained in DMEM/F-12 medium (Gibco BRL) supplemented with 10% fetal bovine serum (FBS), 2 mM glutamine and 50 units/ml penicillin+50 ug/ml streptomycin. The day before transfection, cells were counted and seeded in 6-well plates at a density of 3.5×10⁵ cells per well. Cells were transfected with 1.0 ml 100 nM siRNA complexed with Lipofectamine2000 and analyzed by Northern blotting. Northern blots were hybridised with full-length cDNA probes for mTF and GAPDH in PerfectHyb Plus (Sigma) hybridisation buffer according to the manufacturer's instructions.

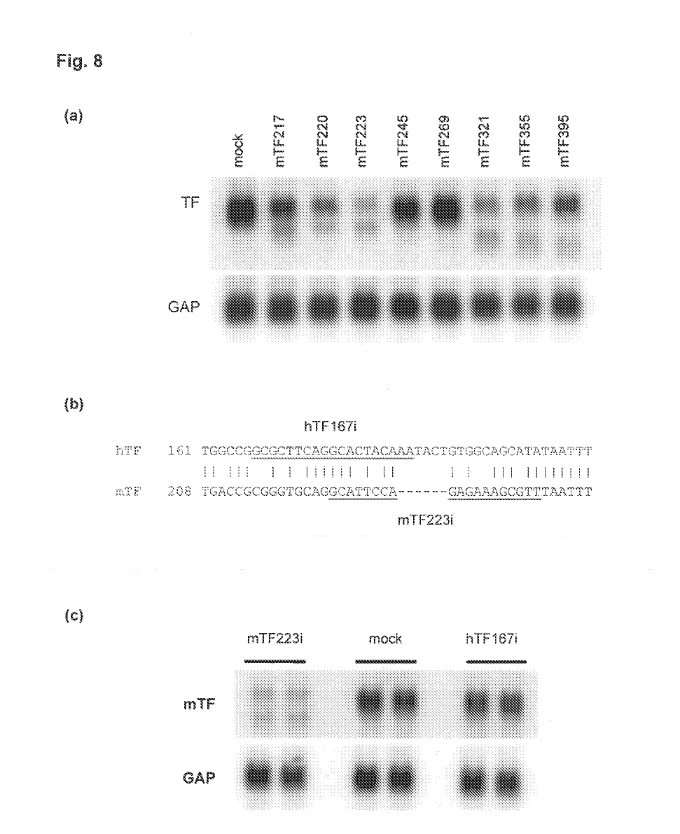

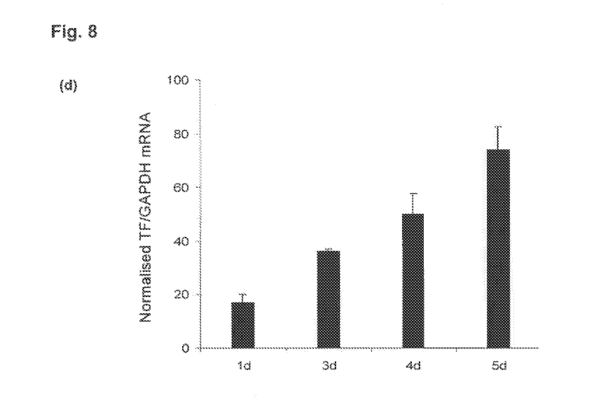

Lipofectamine2000-mediated transfection of B16 cells with 100 nM siRNA demonstrated a highly variable efficiency of the different target sequences (FIG. 8A), consistent with our previous observations. The most effective siRNA, mTF223i, consistently depleted mTF mRNA by approximately 80% 24 h after transfection. This was one of three overlapping siRNA targeted to the region corresponding to the target sequence of the best hTF siRNA, hTF167i. Although highly active against its intended target, the siRNA hTF167i contained multiple mismatches against the mTF sequence (FIG. 8B), and had no effect at all on mTF expression in cultured B16 cells (FIG. 8C). The level of knockdown achieved with mTF223i in cell culture suggested that this siRNA would be a good candidate for in vivo experiments.

We next estimated the duration of TF silencing. For these experiments, cells were sub-cultured every second day following transfection, in order to maintain exponential growth. At the appropriate time points, cells were harvested for mRNA isolation using Dynabeads oligo(dT)₂₅ (Dynal). In a sub-culturing time-course experiment in which cells were maintained in the exponential growth phase, expression of mTF recovered gradually from day 3 to day 5 (FIG. 8D). This window of reduced mTF expression was considered sufficient to attempt in vivo experiments without any further optimization of the siRNA composition (by chemical modification) for increased stability.

### Example 9

### siRNA Toward Murine TF Reduces Circulating Malignant Cells Ability to Form Pulmonary Tumors

As a follow-up to the experiments described in Example 7, we used the eight siRNA specific for murine TF (mTF) to transfect B16 cells. corresponding to the region harbouring the best siRNA targets in Lipofectamine2000-mediated transfection of B16 cells with 100 nM siRNA demonstrated a highly variable efficiency of the different target sequences for the siRNAs used. The two most effective siRNA, mTF223i (SEQ ID NO: 33) and mTF321i (SEQ ID NO: 34), consistently depleted mTF mRNA by 70-80% in cultured B16 cells.

Tail vein injections have been assumed to represent a model for tumor take of blood-borne metastases. We used a well-established model in which tail vein injection of B16-F10 (B16) murine melanoma cells into C57BL/6 mice results in pulmonary colonization within 10-14 days. Experiments were designed and carried out to investigate if knockdown of TF in B16 cells in vitro reduced the tendency for pulmonary metastasis following intra tail vein injection. The day before injection, cells were transfected with a control siRNA against hTF (hTF167i) or with either of two different siRNA targeting mTF (mTF223i, mTF321i). Although highly active against its intended target, the control siRNA hTF167i contained substantial mismatches against mTF, and had no effect at all on mTF expression in cultured B16 cells.

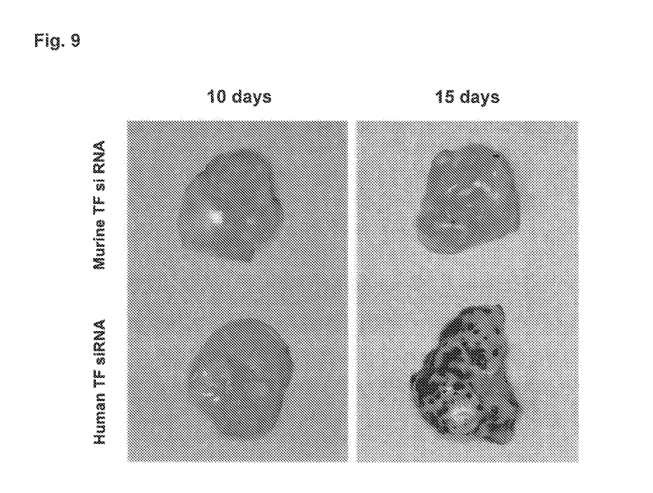

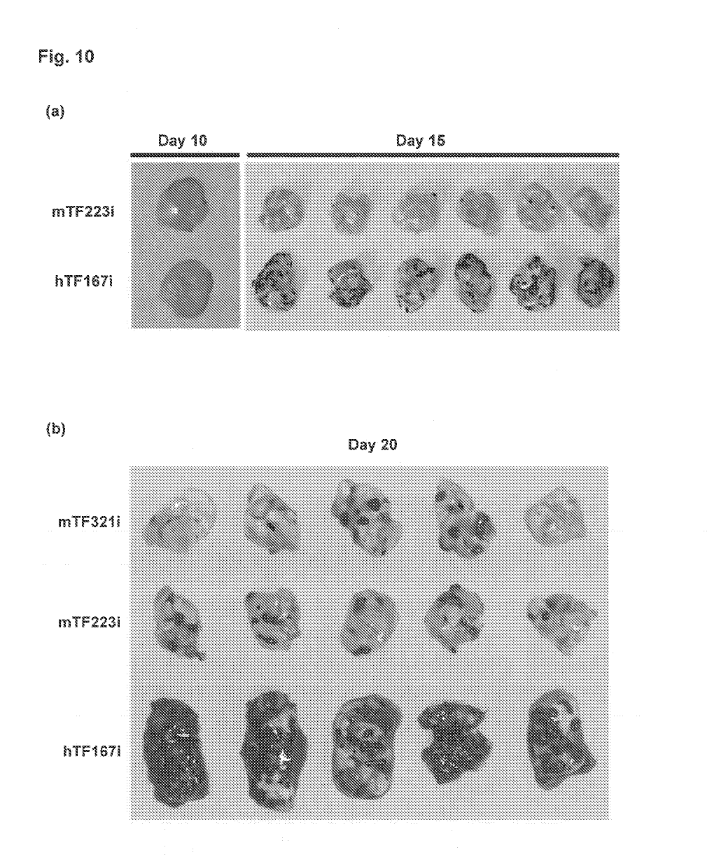

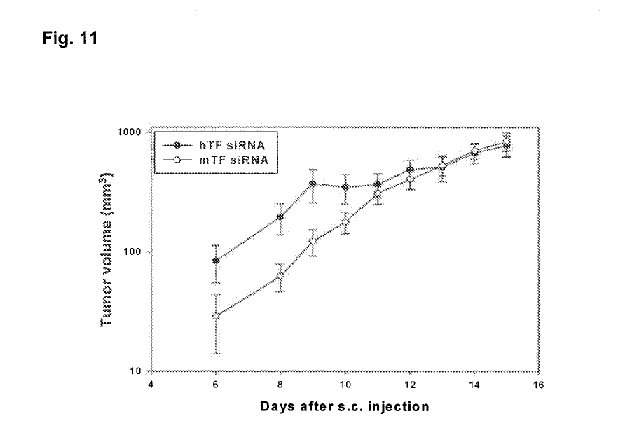

A total of three independent blinded experiments were performed, with at least five mice in each experimental group and harvesting time point. Mice were harvested on day 10 in the first experiment, on days 10 and 15 in the second experiment, and on days 15 and 20 in the third experiment. The data from all experiments are summarized in Table 4. A picture of representative lungs from mice treated from the test and control groups harvested at days 10 and 15 is shown in FIG. 9 and FIG. 10. Both groups of mice that were treated with cells transiently transfected with active (mTF) siRNA, and therefore exhibiting reduced expression of TF, developed significantly less tumors than the control group of mice at all time-points investigated. Thus, our experiments demonstrate that a single liposome-mediated transfection of B16 cells with active mTF siRNA in vitro results in a target-sequence specific delay in development of pulmonary tumors of intravenously injected cells. This is directly attributable to the transient knockdown of TF expression.

The window of protection achieved by the single administration of siRNA in vitro was estimated by observing the survival of mice injected with control- and test-transfected cells. Five or six mice in each group were inspected several times daily and sacrificed at the first indication of tumor-associated stress. The average survival of the mice increased significantly (P=0.01), from 22 days for the control group to 27 days for mice injected with active mTF223i siRNA.

In conclusion, our results clearly demonstrate that TF has a crucial function in promoting lung tumor metastasis of B16 melanoma cells in the C57BL/6 mice. Thus, siRNA directed towards TF is useful in the prevention of metastasis and may be used in the treatment of cancer in mammals. Highly specific TF siRNA molecules adapted to the TF sequence of a specific species may be found by the screening method disclosed in Holen et al. supra, Amarzguioui et al., supra, and Holen et al., supra.

### Example 10

### Growth of Tumors Following siRNA Treatment

The above pulmonary metastasis model cannot delineate which processes in tumor metastasis and progression are influenced by TF. To investigate whether TF had an effect on localized tumor growth rate, we evaluated the growth of tumors following subcutaneous injection of cells transfected with either mTF223i or hTF167i. Each experimental group consisted of 10 mice. The difference between the two growth curves (FIG. 11) indicated a limited and transient delay in the growth of tumors from cells treated with active siRNA compared with control-transfected cells, with a significant difference in tumor size (volume) on day 8 (P=0.03) and day 9 (P=0.05). Upon further incubation, the difference in the average size of tumors in the two groups diminished gradually, so that after day 13 no effect of siRNA was seen. Two different mechanisms likely contribute to the observed growth behaviour. Initially, knockdown of TF expression by siRNA will, to a limited degree, slow down growth of the cells. This window is likely to be relatively short, due to the transient nature of siRNA-mediated silencing. After TF expression has returned to normal, space and nutrient constraints may present the major limitations to continued growth of the tumor, effectively determining its final size.

TABLE 4
Tumor incidence from all experiments.

| siRNA | Day 10 | Day 15 | Day 20 |
|---|---|---|---|
| hTF167i | 107 (n = 12) | >500 (n = 11) | >500 (n = 5) |
| mTF223i | 10* (n = 12) | 33** (n = 11) | 74** (n = 8) |
| mTF321i | n.d. | 16** (n = 5) | 41** (n = 5) |

The average number of tumors and the total number of mice included in the analysis (parentheses) are given for each experimental group and harvest time point. The level of significance of the differences in tumors for test (mTF223i, mTF321i) and control (hTF167i) groups are indicated by asterisks (*: P=0.01, **: P<0.001). n.d.: not determined.

### REFERENCES

1. Bernstein E et al., Role for a bidentate ribonuclease in the initiation step of RNA interference. Nature 409: 363-366 (2001).
2. Camerer E et al., Cell biology of tissue factor, the principal initiator of blood coagulation. Thromb Res. 81: 1-41 (1996).
3. Clemens, J. C. et al., Use of double-stranded RNA interference in Drosophila cell lines to dissect signal transduction pathways. Proc. Natl. Acad. Sci. USA 97: 6499-6503 (2000).
4. Amarzguioui M et al., Secondary structure prediction and in vitro accessibility of mRNA as tools in the selection of target sites for ribozymes. Nuc. Acids Res. 28: 4113-4124 (2000).
5. Hutvagner G et al., A cellular function for the RNA-interference enzyme Dicer in the maturation of the let-7 small temporal RNA. Science 293: 834-838 (2001).
6. Wiiger M T et al., Effects of binding of ligand (FVIIa) to induced tissue factor in human endothelial cells. Thromb Res. 98: 311-321 (2000).
7. Camerer E et al., Opposite sorting of tissue factor in human umbilical vein endothelial cells and Madin-Darby canine kidney epithelial cells. Blood 88: 1339-1349 (1996).
8. Caplen et al., dsRNA-mediated gene silencing in cultured Drosophila cells: a tissue culture model for the analysis of RNA interference. Gene 252: 95-105 (2000).
9. Tuschl T et al., Targeted mRNA degradation by double-stranded RNA in vitro. Genes Dev. 13: 3191-3197 (1999).
10. Elbashir S M et al., Duplexes of 21-nucleotide RNAs mediate RNA interference in cultured mammalian cells. Nature 411: 494-498 (2001).
11. Palauqui J C and Balzergue S. Activation of systemic acquired silencing by localised introduction of DNA. Curr Biol. 9: 59-66 (1999).
12. Fire, A. RNA-triggered gene silencing. Trends Genet. 15: 358-363 (1999).
13. Grishok A et al., Genetic requirements for inheritance of RNAi in C. elegans. Science 287: 2494-2497 (2000).
14. Boutla, A. et al., Short 5′-phosphorylated double-stranded RNAs induce RNA interference in Drosophila. Curr. Biol. 11:1776-1780 (2001).
15. Elbashir, S. M. et al., Functional anatomy of siRNAs for mediating effiecient RNAi in Drosophila melanogaster embryo lysate. EMBO J. 20:6877-6888 (2001).
16. Jacque, J. M. et al., Modulation of HIV-1 replication by RNA interference. Nature 418: 435-438 (2002).
17. Gitlin, L. et al., Short interfering RNA confers intracellular antiviral immunity in human cells. Nature 418:430-434 (2002).
18. Klahre, U. et al., High molecular weight RNAs and small interfering RNAs induce systemic posttranscriptional gene silencing in plants. Proc. Natl. Acad. Sci. USA. 99:11981-11986 (2002).
19. Garrus, J. E. et al., Tsg101 and the vacuolar protein sorting pathway are essential for HIV-1 budding. Cell 107:55-65 (2001).
20. Yu, J. Y. et al., RNA interference by expression of short-interfering RNAs and hairpin RNAs in mammalian cells. Proc. Natl. Acad. Sci. USA, 99:6047-52 (2002).
21. Wilda, M. et al., Killing of leukemic cells with a BCR/ABL fusion gene by RNA interference (RNAi). Oncogene 21:5716-24 (2002).
22. Brummelkamp, T. R. et al., A system for stable expression of short interfering RNAs in mammalian cells. Science 296:550-3 (2002).
23. Paddison, P. J. et al., Short hairpin RNAs (shRNAs) induce sequence-specific silencing in mammalian cells. Genes Dev. 16:948-58 (2002).
24. Nykänen, A. et al, ATP Requirements and Small Interfering RNA Structure in the RNA Interference Pathway. Cell 107:309-321 (2001).
25. Elbashir, S. M. et al., RNA interference is mediated by 21 and 22 nt RNAs. Genes Dev., 15:188-200 (2001).
26. Janowsky, E., and Schwenzer, B. Oligonucleotide facilitators enable a hammerhead ribozyme to cleave long RNA substrates with multiple-turnover activity. Eur. J. Biochem. 254: 129-134 (1998).
27. Billy, E. et al. Specific interference with gene expression induced by long, double-stranded RNA in mouse embryonal teratocarcinoma cell lines. Proc. Natl. Acad. Sci. USA 98:25, 14428-33 (2001).
28. Caplen, N. J. et al. Specific inhibition of gene expression by small double-stranded RNAs in invertebrate and vertebrate systems. Proc. Natl. Acad. Sci. USA 98:17, 9742-47 (2001).
29. Holen, T. et al. Positional effects of short interfering RNAs targeting the human coagulation trigger Tissue Factor, Nucleic Acid Res. 30: 1757-1766 (2000).
30. Tuschl, T. and Borkhardt, A. Small interfering RNAs: a revolutionary tool for the analyses of gene function and gene therapy, Mol. Interv. 2: 158-167 (2002).
31. Harborth et al. Identification of essential genes in cultured mammalian cells using small interfering RNAs, J. Cell Science 114: 4557-4565 (2001).
32. W. Ruf and B. M. Mueller. Tissue Factor in cancer angiogenesis and metastasis. Current Opinion in Hematology 3:379-384 (1996).
33. Hu and Garen. Targeting tissue factor on tumor vascular endothelial cells and tumor cells for immunotherapy in mouse models of prostatic cancer. Proc. Natl. Acad. Sci. 98:10180-12185 (2001).
34. Bromberg et al. Role of tissue factor in metastasis: Functions of the cytoplasmic and extracellular domains of the molecule, Thromb Haemost. 82:88-92 (1999).
35. Toomey et al. Effect of tissue factor deficiency on mouse and tumor development. Proc. Natl. Acad. Sci. 94: 6922-6926 (1997).
36. Zhang et al. J. Clin. Invest. 94:1320-1327 (1994).
37. Amirkhosravi et al. Tissue Factor pathway inhibitor reduces experimental lung metastasis of B16 melanomas. Thromb. Haemost. 87: 930-936 (2002).
38. Ohta et al. Expression of tissue factor in associated with clinical Features and angiogenesis in prostate cancer. Anticancer Research 22:2991-2996 (2002).
39. Bromberg et al. Tissue Factor promotes melanoma metastasis by a pathway independent of blood coagulation. Proc. Natl. Acad. Sci. 92: 8205-8209 (1995).
40. Konigsberg et al. The TF:VIIa complex: clinical significance, structure-function relationship and its role in signaling and metastasis. Thromb, Haemost. 86:757-771 (2001).

### Other Embodiments

From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A short interfering RNA molecule (siRNA), characterized in that it is a single or double stranded siRNA comprising at least 19 nucleotides and which is directed towards a tissue factor (TF) coding nucleic acid sequence or fragments thereof, and wherein the siRNA molecule is selected from the group consisting of
(a) an siRNA molecule having the nucleic acid sequence of SEQ ID NOs: 103-110 and
(b) an siRNA molecule having a sequence which has greater than 90% homology to an siRNA molecule of (a).
